(19) United States
(12) Patent Application Publication
(10) Pub. No.: US 2008/0089941 A1
(43) Pub. Date: Apr. 17, 2008

(54) Fucoidan compositions and methods

(76) Inventor: Thomas E. Mower, Springville, UT (US)

Correspondence Address: ALAN J. HOWARTH, P.O. BOX 1909, SANDY, UT, 84091-1909, US

(21) Appl. No.: 11/809,722

(22) Filed: Jun. 1, 2007

Related U.S. Application Data
(60) Provisional application No. 60/810,233, filed on Jun. 1, 2006.

(51) Int. Cl. A61K 36/00; A61K 31/715; A61K 36/38; A61K 36/42; A61K 36/45; A61K 36/73; A61K 36/736; A61K 36/87; A61K 8/02; A61K 8/73; A61K 9/14; A61P 29/00; A61P 31/00; A61P 35/00; A61P 7/02; A61Q 19/00

(52) U.S. Cl. 424/489; 424/725; 424/732; 424/735; 424/758; 424/765; 424/766; 424/769; 514/54

(57) ABSTRACT

Compositions and methods relating to partially hydrolyzed fucoidan for use in dietary supplements and skin-care products are described. Fucoidan from brown seaweeds is partially hydrolyzed and/or sulfonated and then mixed with other ingredients for use as a dietary supplement in beverage, capsule, or tablet form or for use as a skin-care product. Other ingredients that can be included in the dietary supplements include vitamins, minerals, amino acids, carotenoids, flavonoids, antioxidants, aminosugars, glycosaminoglycans, and botanicals. Skin care products according to the present invention comprise partially hydrolyzed fucoidan and a base.

### CROSS-REFERENCE TO RELATED APPLICATIONS

This application claims the benefit of U.S. Provisional Application No. 60/810,233, filed Jun. 1, 2006, which is hereby incorporated by reference herein in its entirety, including but not limited to those portions that specifically appear hereinafter, the incorporation by reference being made with the following exception: In the event that any portion of the above-referenced provisional application is inconsistent with this application, this application supercedes said above-referenced provisional application.

### STATEMENT REGARDING FEDERALLY SPONSORED RESEARCH OR DEVELOPMENT

Not applicable.

### BACKGROUND OF THE INVENTION

This invention relates generally to dietary supplements incorporating fucoidan derived from seaweed. More particularly, the present invention relates to dietary supplements incorporating fucoidan from seaweed, such as Tongan limu moui and Japanese hoku kombu and mozuku, and optionally including one or more ingredients having a high oxygen radical absorbance capacity (ORAC).

Fucoidan

Fucoidan is a sulfated polysaccharide found in many sea plants and animals and is particularly concentrated in the cell walls of brown algae (Phaeophyceae). Fucoidan is a complex carbohydrate polymer composed mostly of sulfated L-fucose residues. These polysaccharides are easily extracted from the cell wall of brown algae with hot water or dilute acid and can account for more than 40% of the dry weight of isolated cell walls. O. Berteau & B. Mulloy, Sulfated fucans, fresh perspectives: structures, functions, and biological properties of sulfated fucans and an overview of enzymes active toward this class of polysaccharide, 13 Glycobiology 29R-40R (2003). Fucoidan structure appears to be linked to algal species, but there is insufficient evidence to establish any systematic correspondence between structure and algal order. High amounts of α(1-3) and α(1-4) glycosidic bonds occur in fucoidans from Ascophyllum nodosum. A disaccharide repeating unit of alternating α(1-3) and α(1-4) bonds represents the most abundant structural feature of fucoidans from both A. nodosum and Fucus vesiculosus. Sulfate residues are found mainly in position 4. Further heterogeneity is added by the presence of acetyl groups coupled to oxygen atoms and branches, which are present in all the plant fucoidans.

Fucoidan-containing seaweeds have been eaten and used medicinally for at least 3000 years in Tonga and at least 2000 years in China. An enormous amount of research has been reported in the modern scientific literature, where more than 500 studies are referenced in a PubMed search for fucoidan.

The physiological properties of fucoidans in the algae appear to be a role in cell wall organization and possibly in cross-linking of alginate and cellulose and morphogenesis of algal embryos. Fucoidans also have a wide spectrum of activity in biological systems. They have anticoagulant and antithrombotic activity, act on the inflammation and immune systems, have antiproliferative and antiadhesive effects on cells, and protect cells from viral infection.

Further, fucoidan has numerous beneficial functions that heal and strengthen different systems of the body, including anti-viral, anti-inflammatory, anti-coagulant, and anti-tumor properties. A. I. Usov et al., Polysaccharides of Algae: Polysaccharide Composition of Several Brown Algae from Kamchatka, 27 Russian J. Bio. Chem. 395-399 (2001). Fucoidan has been found to build and stimulate the immune system. Research has also indicated that fucoidan reduces allergies, inhibits blood clotting, fights diabetes by controlling blood sugar, prevents ulcers, relieves stomach disorders, reduces inflammation, protects the kidneys by increasing renal blood flow, and detoxifies the body. Fucoidan also helps to reduce and prevent cardiovascular disease by lowering high cholesterol levels and activating enzymes involved in the beta-oxidation of fatty acids.

A Japanese study found that fucoidans enhanced phagocytosis, the process in which white blood cells engulf, kill, digest, and eliminate debris, viruses, and bacteria. An American study reported that fucoidans increased the number of circulating mature white blood cells. An Argentine study and a Japanese study found that fucoidans inhibited viruses, such as herpes simplex type I, from attaching to, penetrating, and replicating in host cells. A Swedish study is among the many that showed fucoidans inhibit inflammation cascades and tissue damage that may lead to allergies. Other studies, such as one in Canada, found that fucoidans block the complement activation process that is believed to play an adverse role in chronic degenerative diseases, such as atherosclerosis, heart attack, and Alzheimer's disease. Two American studies found that fucoidans increase and mobilize stem cells.

Researchers have also determined that fucoidan tends to combat cancer by reducing angiogenesis (blood vessel growth), inhibiting metastasis (spreading of cancer cells to other parts of the body), and promoting death of cancer cells. Certain societies that make brown seaweed part of their diet appear to have remarkably low instances of cancer. For example, the prefecture of Okinawa, where the inhabitants enjoy some of the highest life expectancies in Japan, also happens to have one of the highest per capita consumption rates of fucoidans. It is noteworthy that the cancer death rate in Okinawa is the lowest of all the prefectures in Japan.

Brown seaweed is found in abundance in various ocean areas of the world. One of the purest locations that provides some of the highest yields of fucoidan is in the clear waters surrounding the Tongan islands, where the seaweed is called limu moui. In Japan, hoku kombu (Laminaria japonica), is said to be particularly rich in fucoidans and is similar to limu moui. The Japanese also consume at least two other types of brown seaweed-wakame and mozuku (Cladosiphon and Nemacystus).

Typically, about four percent by weight of Tongan limu moui is fucoidan. There are at least three types of fucoidan polymer molecules found in brown seaweed. U-fucoidan, having about 20 percent glucuronic acid, is particularly active in carrying out cancer cell destruction. F-fucoidan, a polymer of mostly sulfated fucose, and G-fucoidan both tend to induce the production of HGF cells that assist in restoring and repairing damaged cells. All three types of fucoidan also tend to induce the production of agents that strengthen the immune system.

Accordingly, consumable beverages and other compositions of fucoidan are needed to benefit from the many advantages mentioned above. Methods of preparation of fucoidan may be used to enhance consumption while not destroying its beneficial effects.

Skin

The skin is made up of two major layers. The epidermis is the top layer and forms a protective covering for skin and controls the flow of water and substances in and out of the skin. To stay healthy, the skin has to cope with changing environmental conditions and repair damage at the same time. The skin is in a constant state of repair as it sheds the dead cells on the surface and replenishes the lower layers. The dermis is the lower level of the skin and is the layer that provides the strength, elasticity, and thickness to the skin. Cells in the dermis are responsible for synthesis and secretion of all the dermal matrix components, such as collagen, elastin, and glycosaminoglycans. Collagen provides the strength, elastin the elasticity, and glycosaminoglycans the moistness and plumpness of the skin.

The skin may be abused by soaps, emulsifier-based cosmetics, hot water, or organic solvents, for example. These each contribute to rob the skin of essential moisture, and to create a stressed barrier that does not function properly. Moisture loss and irritation increases, leaving the skin sensitive, scaly, and dry. Free-radical activity multiplies, causing more wrinkles and premature aging.

Furthermore, the skin is subject to deterioration through dermatological disorders, environmental abuse, such as from wind, air conditioning, and central heating, or through the normal aging process, which may be accelerated by exposure of skin to sun. The thickness of the dermal layer is reduced due to aging, thus causing the skin to slacken. This is believed to be partially responsible for the formation of wrinkles. In recent years, the demand for cosmetic compositions and cosmetic methods for improving the appearance and condition of skin has grown enormously.

Consumers are increasingly seeking anti-aging cosmetic products that treat or delay the visible signs of actual aging and weathered skin, such as wrinkles, lines, sagging, hyper-pigmentation, and age spots. Consumers also frequently seek other benefits from cosmetic products in addition to anti-aging. The concept of sensitive skin has raised the demand for cosmetic products that improve the appearance and condition of sensitive, dry, and flaky skin and soothe red or irritated skin. Consumers also desire cosmetic products that treat spots, pimples, blemishes, and so forth.

Research shows that using a skin care product that includes the skin's natural building blocks speeds the skin's ability to repair itself and keeps the barrier function of skin at optimal levels. This approach treats the problem, not merely the symptom. Irritation stops before it can start, so recurring problems are avoided, thus bringing the skin back to ideal conditions.

Consumer demand for natural-based products has been growing in recent years. Chemical synthesis is perceived as environmentally unsafe. A chemically synthesized ingredient may contain harsh chemicals. Natural products are perceived as pure and mild and superior to chemically synthesized products. Delivering a cosmetic benefit from plant sources, however, is not trivial. To derive a real benefit from a natural source, not only does a plant or a part of the plant containing a specific active ingredient have to be identified, but a minimum concentration and/or a specific extract of that plant has to be identified that truly delivers a cosmetic benefit.

Accordingly, consumers demand an effective treatment for the skin and wrinkles that moisturizes, heals, and soothes the vulnerable and delicate surface of the skin. Further, consumers demand that treatment for the skin be based on natural products to promote healing and preserve youthful appearance.

In view of the foregoing, it will be appreciated that providing a fucoidan-containing nutritional supplements and skin-care products would be a significant advancement in the art.

### BRIEF SUMMARY OF THE INVENTION

An illustrative embodiment of the present invention comprises compositions of matter comprising mixtures or compounds of partially hydrolyzed and/or sulfonated fucoidan and one or more antioxidants. Illustrative antioxidants, without limitation, include superoxide dismutase, astaxanthin, curcumin, curcuminoids, vitamin E, raspberry, blueberry, pomegranate, tocopherols, green tea, white tea, dark chocolate, chocolate, cocoa, spirulina, bromelain, vitamin C, rutin, grape seed extract, pycnogenols, oligomeric proanthocyanidins, anthocyanidins, procyanidins, selenium, beta-carotene, zinc, bilberry, cranberry, polyphenols, flavones, strawberry, ellagic acid, coumarin, ferulic acid, resveratrol, alpha-lipoic acid, tomatoes, avocados, broccoli, lycopene, lutein, vitamin A, folic acid, folates, carotenoids, olive leaf extract, ground cloves, ground cinnamon, oregano, blackberry, black currant, polyphenolics, bioflavonoids, flavonoids, flavanols, catechols, goji, tamarind, mangosteen, xanthones, tart cherries, cherries, asparagus, glutathione, catechins, epicatechins, plums, ruby queen plum, kiwi fruit, Ganoderma lucidum, thiols, onions, apples, red cabbage, star fruit, carambola, white pine bark extract, N-acetyl cysteine, citrus, and beta-cryptoxanthin.

Another illustrative embodiment of the present invention comprises methods of making compositions of matter comprising mixtures or compounds of partially hydrolyzed and/or sulfonated fucoidan and one or more antioxidants, the methods comprising mixing the partially hydrolyzed and/or sulfonated fucoidan and the one or more antioxidants.

Still another illustrative embodiment of the present invention comprises methods of using compositions of matter comprising mixtures or compounds of partially hydrolyzed and/or sulfonated fucoidan and one or more antioxidants, the methods comprising administering the mixtures or compounds of partially hydrolyzed and/or sulfonated fucoidan and one or more antioxidants to an individual.

A still further illustrative embodiment of the present invention comprises compositions of matter for delivery of partially hydrolyzed and/or sulfonated fucoidan, the compositions comprising said fucoidan formulated as nanoparticles.

Yet another illustrative embodiment of the present invention comprises a method of making compositions of matter for delivery of partially hydrolyzed and/or sulfonated fucoidan, the method comprising formulating the fucoidan as nanoparticles.

Another illustrative embodiment of the invention comprises a method of using compositions of matter comprising partially hydrolyzed and/or sulfonated fucoidan formulated as nanoparticles, the method comprising administering the nanoparticles to an individual.

Another illustrative embodiment of the present invention comprises a composition of matter for delivery of partially hydrolyzed and/or sulfonated fucoidan wherein the composition comprises a mixture of the fucoidan and structured water or clustered water.

Still another illustrative embodiment of the invention comprises a method of making compositions of matter for delivery of partially hydrolyzed and/or sulfonated fucoidan, the method comprising mixing the fucoidan and structured water or clustered water.

Yet another illustrative embodiment of the invention comprises a method of using a composition of matter comprising a mixture of partially hydrolyzed and/or sulfonated fucoidan and structured water or clustered water, the method comprising administering said mixture to an individual.

Another illustrative embodiment of the invention comprises a composition of matter comprising mixtures or compounds of partially hydrolyzed and/or sulfonated fucoidan and one or more peptides or polypeptides.

Still another illustrative embodiment of the invention comprises a method of making a composition of matter comprising mixtures or compounds of partially hydrolyzed and/or sulfonated fucoidan and one or more peptides or polypeptides, the method comprising mixing or reacting the partially hydrolyzed and/or sulfonated fucoidan and one or more peptides or polypeptides to result in said composition.

Yet another illustrative embodiment of the invention comprises a method of using compositions of matter comprising mixtures or compounds of partially hydrolyzed and/or sulfonated fucoidan and one or more peptides or polypeptides, the method comprising administering said composition to an individual.

Another illustrative embodiment of the present invention comprises a composition of matter for treating arthritis and/or strengthening of joints and cartilage, the composition comprising mixtures or compounds of partially hydrolyzed and/or sulfonated fucoidan and an agent for treating arthritis and/or strengthening joints and cartilage. An illustrative agent for treating arthritis and/or strengthening joints and cartilage includes a member selected from the group consisting of one or more members selected from the group consisting of glucosamine sulfate, glucosamine HCl, glucosamine phosphate, acetyl glucosamine, shark cartilage, chondroitin sulfate, galactolipids, wool keratin protein extract, keratin extract, hyaluronic acid, stinging nettle, glucomannan, type 11 collagen, collagen hydrolysate, and mixtures thereof.

Still another illustrative embodiment of the present invention comprises a method for making a composition of matter for treatment of arthritis and/or strengthening of joints and cartilage comprising mixtures or compounds of partially hydrolyzed and/or sulfonated fucoidan and an agent for treating arthritis and/or strengthening joints and cartilage, the method comprising mixing or reacting said partially hydrolyzed and/or sulfonated fucoidan and said agent for treating arthritis and/or strengthening joints and cartilage to result in said composition.

Yet another illustrative embodiment of the present invention comprises a method for treating arthritis and/or strengthening of joints and cartilage, the method comprising administering a composition of matter comprising a mixture or compound of partially hydrolyzed and/or sulfonated fucoidan and an agent for treating arthritis and/or strengthening joints and cartilage.

Another illustrative embodiment of the present invention comprises a composition of matter for strengthening the immune system, the composition comprising mixtures or compounds of partially hydrolyzed and/or sulfonated fucoidan and an agent for strengthening the immune system. An illustrative agent for strengthening the immune system is a member selected from the group consisting of noni, mangosteen, and mixtures thereof.

Still another illustrative embodiment of the present invention comprises a method for making a composition of matter for strengthening the immune system, the method comprising mixing or reacting partially hydrolyzed and/or sulfonated fucoidan and an agent for strengthening the immune system to result in a mixture or compound.

Yet another illustrative embodiment of the present invention comprises a method for strengthening the immune system, the method comprising administering a compositions of matter comprising a mixture or compound of partially hydrolyzed and/or sulfonated fucoidan and an agent for strengthening the immune system.

Another illustrative embodiment of the present invention comprises a composition of matter comprising mixtures or compounds of partially hydrolyzed and/or sulfonated fucoidan and a cell signaling agent. An illustrative cell signaling agent is a member selected from the group consisting of bitter orange, caffeine, taurine, green coffee bean, and mixtures thereof.

Still another illustrative embodiment of the present invention comprises a method for making a composition of matter comprising a mixture or compound of partially hydrolyzed and/or sulfonated fucoidan and a cell signaling agent, the method comprising mixing or reacting said partially hydrolyzed and/or sulfonated fucoidan and said cell signaling agent to result in a mixture or compound.

Yet another illustrative embodiment of the present invention comprises a method of using a composition of matter comprising mixtures or compounds of partially hydrolyzed and/or sulfonated fucoidan and a cell signaling agent, the method comprising administering the composition to an individual.

Another illustrative embodiment of the present invention comprises an energy drink composition comprising mixtures or compounds of partially hydrolyzed and/or sulfonated fucoidan, water, and an energy enhancing agent. An illustrative energy enhancing agent is one or more saccharides selected from the group consisting of glucose, sucrose, fructose, and mixtures thereof.

Still another illustrative embodiment of the present invention comprises a method for making an energy drink composition comprising mixtures or compounds of partially hydrolyzed and/or sulfonated fucoidan, water, and an energy enhancing agent, the method comprising mixing or reacting said partially hydrolyzed and/or sulfonated fucoidan, water, and said energy enhancing agent to result in said composition.

Yet another illustrative embodiment of the present invention comprises a method of using an energy drink composition comprising mixtures or compounds of partially hydrolyzed and/or sulfonated fucoidan, water, and an energy enhancing agent, the method comprising administering said composition to an individual.

Another illustrative embodiment of the present invention comprises a composition of matter comprising mixtures or compounds of partially hydrolyzed and/or sulfonated fucoidan and a heart strengthening agent. An illustrative heart strengthening agent comprises wolfberry.

Still another illustrative embodiment of the present invention comprises a method for making a composition of matter comprising mixtures or compounds of partially hydrolyzed and/or sulfonated fucoidan and a heart strengthening agent, the method comprising mixing or reacting said partially hydrolyzed and/or sulfonated fucoidan and said heart strengthening agent to result in said composition.

Yet another illustrative embodiment of the present invention comprises a method of strengthening the heart, the method comprising administering a composition of matter comprising mixtures or compounds of partially hydrolyzed and/or sulfonated fucoidan and a heart strengthening agent to an individual.

Another illustrative embodiment of the present invention comprises a composition of matter comprising mixtures or compounds of partially hydrolyzed and/or sulfonated fucoidan and an agent for reversing muscle loss, increasing muscle mass, and or bone density. An illustrative agent for reversing muscle loss, increasing muscle mass, and or bone density comprises one or more α-amino acids or salts or esters thereof.

Still another illustrative embodiment of the present invention comprises a method for making a composition of matter comprising mixtures or compounds of partially hydrolyzed and/or sulfonated fucoidan and an agent for reversing muscle loss, increasing muscle mass, and or bone density, the method comprising mixing or reacting said partially hydrolyzed and/or sulfonated fucoidan and said agent for reversing muscle loss, increasing muscle mass, and or bone density to result in said composition.

Yet another illustrative embodiment of the present invention comprises a method of reversing muscle loss and increasing muscle mass and/or bone density, the method comprising administering a composition of matter comprising mixtures or compounds of partially hydrolyzed and/or sulfonated fucoidan and an agent for reversing muscle loss, increasing muscle mass and/or bone density to an individual.

Another illustrative embodiment of the present invention comprises a composition of matter for increasing bone density, regulating prostate function, and/or treating post- and pre-menopausal conditions, said composition comprising mixtures or compounds of partially hydrolyzed and/or sulfonated fucoidan and an agent for increasing bone density, regulating prostate function, and/or treating post- and pre-menopausal conditions. An illustrative agent for increasing bone density, regulating prostate function, and/or treating post- and pre-menopausal conditions comprises equol.

Still another illustrative embodiment of the present invention comprises a method for making a composition of matter for increasing bone density, regulating prostate function, and/or treating post- and pre-menopausal conditions, said method comprising mixing or reacting partially hydrolyzed and/or sulfonated fucoidan and an agent for increasing bone density, regulating prostate function, and/or treating post- and pre-menopausal conditions to result in a mixture or compound.

Yet another illustrative embodiment of the present invention comprises a method for increasing bone density, regulating prostate function, and/or treating post- and pre-menopausal conditions, the method comprising administering a composition of matter comprising mixtures or compounds of partially hydrolyzed and/or sulfonated fucoidan and an agent for increasing bone density, regulating prostate function, and/or treating post- or pre-menopausal conditions to an individual.

Another illustrative embodiment of the present invention comprises a composition of matter for treating cancer comprising mixtures or compounds of partially hydrolyzed and/or sulfonated fucoidan and an agent for treating cancer. An illustrative agent for treating cancer is a member selected from the group consisting of capsican, lycopene, lutein, perillyl oil, cranberry, curcumin, turmeric, and mixtures thereof.

Still another illustrative embodiment of the present invention comprises a method for making a composition of matter for treating cancer, the method comprising mixing or reacting partially hydrolyzed and/or sulfonated fucoidan and an agent for treating cancer to result in a mixture or compound.

Yet another illustrative embodiment of the present invention comprises a method for treating cancer, the method comprising administering a compositions of matter comprising mixtures or compounds of partially hydrolyzed and/or sulfonated fucoidan and an agent for treating cancer to an individual.

Another illustrative embodiment of the present invention comprises a composition of matter for treatment of post- and pre-menopausal conditions, the composition comprising mixtures or compounds of partially hydrolyzed and/or sulfonated fucoidan and an agent for treating post-menopausal and pre-menopausal conditions. An illustrative agent for treating post-menopausal and pre-menopausal conditions comprises one or more isoflavones.

Still another illustrative embodiment of the present invention comprises a method for making a composition of matter for treatment of post- and pre-menopausal conditions, the method comprising mixing or reacting partially hydrolyzed and/or sulfonated fucoidan and an agent for treating post-menopausal and pre-menopausal conditions to result in a mixture or compound.

Yet another illustrative embodiment of the present invention comprises a method for treating post- and pre-menopausal conditions, the method comprising administering a compositions of matter comprising mixtures or compounds of partially hydrolyzed and/or sulfonated fucoidan and an agent for treating post-menopausal and pre-menopausal conditions to an individual.

Another illustrative embodiment of the present invention comprises a composition for treating stretch marks and scars on skin, the composition comprising mixtures or compounds comprising partially hydrolyzed and/or sulfonated fucoidan and an agent for treating stretch marks and scars on skin.

Still another illustrative embodiment of the present invention comprises a method of making compositions of matter for treatment of stretch marks and scars on skin, the method comprising mixing or reacting partially hydrolyzed and/or sulfonated fucoidan and an agent for treating stretch marks and scars on skin to result in a mixture or compound.

Yet another illustrative embodiment of the present invention comprises a method for treating stretch marks and scars on skin, the method comprising contacting the affected area with a composition comprising partially hydrolyzed and/or sulfonated fucoidan and an agent for treating stretch marks and scars on skin.

Still another illustrative embodiment of the present invention comprises a composition for extending life, counteracting aging processes, and activating youth-extending biosystems, the composition comprising mixtures or compounds of partially hydrolyzed and/or sulfonated fucoidan and an agent for extending life, counteracting aging processes, and activating youth-extending biosystems.

Yet another illustrative embodiment of the present invention comprises a method of making compositions for extending life, counteracting aging processes, and activating youth-extending biosystems, the method comprising mixing partially hydrolyzed and/or sulfonated fucoidan and an agent for extending life, counteracting aging processes, and activating youth-extending biosystems to result in a mixture or compound.

Another illustrative embodiment of the present invention comprises a method for extending life, counteracting aging processes, and activating youth-extending biosystems, the method comprising administering a composition comprising a mixture or compound comprising partially hydrolyzed and/or sulfonated fucoidan and an agent for extending life, counteracting aging processes, and activating youth-extending biosystems to an individual.

### DETAILED DESCRIPTION

Before the present fucoidan-containing compositions and methods are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior invention.

It must be noted that, as used in this specification and the appended claims, the singular forms “a,” “an,” and “the” include plural referents unless the context clearly dictates otherwise. Thus, for example, reference to a dietary supplement containing “a partially hydrolyzed fucoidan” includes a mixture of two or more of such partially hydrolyzed fucoidans, reference to “an acid” includes reference to two or more of such acids, and reference to “a preservative” includes reference to a mixture of two or more of such preservatives.

In describing and claiming the present invention, the following terminology will be used in accordance with the definitions set out below.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

As used herein, “partially hydrolyzed fucoidan” means fucoidan that has been hydrolyzed into smaller polymers and oligomers, but not so thoroughly hydrolyzed as to result in complete hydrolysis to monosaccharides.

As used herein, “high ORAC value” or similar terms means an ORAC value of at least about 400 per 100 grams of fruit or vegetable. For example, blueberries have an ORAC value of about 2,400 per 100 grams, and the following fruits have ORAC values as shown in parentheses per 100 grams: blackberries (2,036), cranberries (1,750), strawberries (1,540), raspberries (1,220), plums (949), oranges (750), red grapes (739) cherries (670), kiwi fruit (602), and white grapes (446). Other fruits known to have a high ORAC value include black grapes, mangosteen, noni, aronia, wolfberry, and acai, and the like. Further, nutraceutical ingredients known to have high ORAC values include proanthocyanidins, such as from extracts of grape seed and bark of white pine of southern Europe (e.g., pycnogenol, U.S. Pat. No. 4,698,360), and curcuminoids. Oligomeric proanthocyanidins (OPC) are illustrative.

As used herein, “sterilizing” and similar terms means, with respect to nutritional supplements having a pH less than 4.6 and a water activity greater than 0.85, pasteurizing the nutritional supplement and storing at room temperature. With respect to nutritional supplements having a pH greater than 4.6 and a water activity greater than 0.85, “sterilizing” and similar terms mean applying heat such that the nutritional supplement is rendered free of microorganisms capable of reproducing in the nutritional supplement under normal non-refrigerated conditions of storage and distribution.

As used herein, “pasteurization” traditionally means a process named after scientist Louis Pasteur by which every particle of milk is heated to not lower than 62.8° C. (i.e., 145° F.) for not less than 30 minutes and promptly cooled to destroy any harmful bacteria that may be present without affecting flavor and food value. Currently, the most common method of pasteurization in the United States is High Temperature Short Time (HTST) pasteurization, which uses metal plates and hot water to raise temperatures to 71.7° C. (i.e., 161° F.) for not less than 15 seconds, followed by rapid cooling. Ultra Pasteurization (UP) is a process similar to HTST pasteurization, but using higher temperatures and longer times. UP pasteurization results in a product with longer shelf life but still requiring refrigeration of milk, but not of acidified foods or nutritional supplements (pH<4.6). Another method, Ultra High Temperature (UHT) pasteurization, raises the temperature to over 93.3° C. (i.e., 200° F.) for a few seconds, followed by rapid cooling. A UHT-pasteurized product that is packaged aseptically results in a “shelf stable” product that does not require refrigeration until it is opened.

As used herein, “aseptic processing and packaging” and similar terms mean the filling of a sterilized cooled product into pre-sterilized containers, followed by aseptic hermetic sealing, with a pre-sterilized closure, in an atmosphere free of microorganisms.

As used herein, “hermetically sealed container” and similar terms mean a container that is designed and intended to be secure against the entry of microorganisms and thereby to maintain the sterility of its contents after processing.

As used herein, “tablets” are solid dosage forms containing a dietary supplement with or without suitable excipients or diluents and prepared either by compression or molding methods well known in the art. Tablets have been in widespread use since the latter part of the 19^{th} century and their popularity continues. Tablets remain popular as a dosage form because of the advantages afforded both to the manufacturer (e.g., simplicity and economy of preparation, stability, and convenience in packaging, shipping, and dispensing) and the user (e.g., accuracy of dosage, compactness, portability, blandness of taste, and ease of administration). Although tablets are most frequently discoid in shape, they may also be round, oval, oblong, cylindrical, or triangular. They may differ greatly in size and weight depending on the amount of dietary supplement present and the intended method of administration. They are divided into two general classes, (1) compressed tablets, and (2) molded tablets or tablet triturates. In addition to the active or therapeutic ingredient or ingredients, tablets contain a number or inert materials or additives. A first group of such additives includes those materials that help to impart satisfactory compression characteristics to the formulation, including diluents, binders, and lubricants. A second group of such additives helps to give additional desirable physical characteristics to the finished tablet, such as disintegrators, colors, flavors, and sweetening agents.

As used herein, “diluents” are inert substances added to increase the bulk of the formulation to make the tablet a practical size for compression. Commonly used diluents include calcium phosphate, calcium sulfate, lactose, kaolin, mannitol, sodium chloride, dry starch, powdered sugar, silica, and the like.

As used herein, “binders” are agents used to impart cohesive qualities to the powdered material. Binders, or “granulators” as they are sometimes known, impart a cohesiveness to the tablet formulation, which insures the tablet remaining intact after compression, as well as improving the free-flowing qualities by the formulation of granules of desired hardness and size. Materials commonly used as binders include starch; gelatin; sugars, such as sucrose, glucose, dextrose, molasses, and lactose; natural and synthetic gums, such as acacia, sodium alginate, extract of Irish moss, panwar gum, ghatti gum, mucilage of isapol husks, carboxymethylcellulose, methylcellulose, polyvinylpyrrolidone, Veegum, microcrystalline cellulose, microcrystalline dextrose, amylose, and larch arabogalactan, and the like.

As used herein, “lubricants” are materials that perform a number of functions in tablet manufacture, such as improving the rate of flow of the tablet granulation, preventing adhesion of the tablet material to the surface of the dies and punches, reducing interparticle friction, and facilitating the ejection of the tablets from the die cavity. Commonly used lubricants include talc, magnesium stearate, calcium stearate, stearic acid, and hydrogenated vegetable oils.

As used herein, “disintegrators” or “disintegrants” are substances that facilitate the breakup or disintegration of tablets after administration. Materials serving as disintegrants have been chemically classified as starches, clays, celluloses, algins, or gums. Other disintegrators include Veegum HV, methylcellulose, agar, bentonite, cellulose and wood products, natural sponge, cation-exchange resins, alginic acid, guar gum, citrus pulp, cross-linked polyvinylpyrrolidone, carboxymethylcellulose, and the like.

As used herein, “coloring agents” are agents that give tablets a more pleasing appearance, and in addition help the manufacturer to control the product during its preparation and help the user to identify the product. Any of the approved certified water-soluble FD&C dyes, mixtures thereof, or their corresponding lakes may be used to color tablets. A color lake is the combination by adsorption of a water-soluble dye to a hydrous oxide of a heavy metal, resulting in an insoluble form of the dye.

As used herein, “flavoring agents” vary considerably in their chemical structure, ranging from simple esters, alcohols, and aldehydes to carbohydrates and complex volatile oils. Natural and synthetic flavors of almost any desired type are now available.

As used herein, “capsules” are solid dosage forms in which the dietary supplement is enclosed in a hard or soft (including gel caps), soluble container or shell of a suitable polymer, such as gelatin. The soft gelatin capsule was invented by Mothes, a French pharmacist in 1833. During the following year DuBlanc obtained a patent for his soft gelatin capsules. In 1848 Murdock patented the two-piece hard gelatin capsule. The encapsulation of medicinal agents, dietary supplements, and the like remains a popular method of administering agents by the oral route. Capsules are tasteless, easily administered, and easily filled. Some persons find it easier to swallow capsules than tablets, therefore preferring to take this form when possible. This preference has prompted manufacturers to market products in capsule form even though the product has already been produced in tablet form.

As used herein, “pharmaceutical necessities” means substances that are of little or no dietary or therapeutic value, but which are useful in the manufacture and compounding of various dietary supplement preparations. These substances include antioxidants and preservatives; coloring, flavoring, and diluting agents; emulsifying and suspending agents; ointment bases; pharmaceutical solvents; and miscellaneous agents. See, for example, Remington's Pharmaceutical Sciences for a review of what is known in the art concerning pharmaceutical necessities.

As used here, “powders” means a solid dosage form intended to be suspended or dissolved in water or another liquid or mixed with soft foods prior to administration. Powders are typically prepared by spray drying or freeze drying of liquid formulations. Powders are advantageous due to flexibility, stability, rapid effect, and ease of administration.

As used herein, “Brix” is a scale for measuring the sugar content of grapes, wine, and the like. Each degree of Brix is equivalent to one gram of sugar per 100 ml of liquid. Thus, an 18 degree Brix sugar solution contains 18% by weight of sugar. Brix also describes the percent of suspended solids in a liquid. Thus, 95 Brix, for example, denotes a liquid that contains 95% by weight of suspended solids. Brix is measured with an optical device called a refractometer. The Brix system of measurement is named for A. F. W. Brix, a 19^{th} century German inventor.

As used herein, “glucosamine” means glucosamine, salts thereof such as glucosamine sulfate or glucosamine succinate, derivatives thereof such as N-acetylglucosamine, and mixtures thereof.

As used herein, “chondroitin” means chondroitin, salts thereof such as chondroitin sulfate, esters thereof, and mixtures thereof.

As used herein, “cartilage protector” means a precursor in the synthesis of cartilage, such as glucosamine or chondroitin.

As used herein, “vitamin D” includes all of its active forms including, for example, vitamin D₂ (ergocalciferol), vitamin D₃ (cholecalciferol), and mixtures thereof. Similarly, “vitamin E” includes all of its active forms including, for example, alpha-tocopherol, beta-tocopherol, gamma-tocopherol, delta-tocopherol, and mixtures thereof. Similarly, “vitamin A” includes all of its active forms including, for example vitamin A₁ (retinol), vitamin A₂ (dehydroretinol), vitamin A acid (retinoic acid), and mixtures thereof. Similarly, “vitamin K” includes all of its active forms including, for example, vitamin K₁ (phylloquinone), vitamin K₂ (farnoquinone), vitamin K₃ (menadione or menaquinone), vitamins K₄₋₇ (synthetic analogs of menadione), and mixtures thereof. Similarly, “vitamin B-12” includes all of its active forms including, for example, cyanocobalamin, methylcobalamin, hydroxocobalamin, nitritocobalamin, and mixtures thereof.

As used herein, “derivatives” of vitamins means alternative, biologically active forms of a particular vitamin. For example, derivatives of vitamin E include esters of vitamin E, such as d-alpha-tocopheryl acetate. As another example, derivatives of vitamin A include esters of vitamin A, such as retinyl palmitate. As still another example, derivatives of niacin include niacinamide. As yet another example, derivatives of pyridoxine include pyridoxal and pyridoxamine. As a still further example, derivatives of vitamins that are acids include salts of such acids, for example, calcium ascorbate, thiamine hydrochloride, pyridoxine hydrochloride, calcium pantothenate, and the like.

As used herein, “effective amount” means an amount of a component of the dietary supplement that is nontoxic but sufficient to provide the desired effect and performance at a reasonable benefit/risk ratio attending any dietary supplement. For example, an effective amount of a vitamin or mineral is an amount sufficient to prevent a deficiency thereof or to reduce the incidence of some cancers, i.e., lung (vitamin E, folic acid, vitamin D, selenium), prostate (vitamin E, vitamin D, selenium), stomach (vitamin C), colorectal (folic acid, vitamin D, selenium), skin (selenium), cervix (folic acid), and breast (vitamin D); osteoporosis (vitamin D, vitamin K, calcium, magnesium, vanadium, and possibly boron and copper); osteoarthritis (calcium); macular degeneration or cataracts (riboflavin, vitamin C, vitamin E, selenium); heart disease (vitamin E, folic acid, pyridoxine, vitamin A, magnesium, selenium, copper); neurologic disease (thiamine, niacin, pantothenic acid, folic acid, vitamin B-12); or Alzheimer's disease (vitamin E), or to aid in regeneration of connective tissue (vitamin C, copper, iron, manganese, zinc). An effective amount of a carotenoid is an amount sufficient to provide a beneficial effect, such as reduce the incidence of some cancers, i.e., skin and mucous membranes (β-carotene), digestive tract (β-carotene, lycopene), prostate and stomach (lycopene), lung (lutein); macular degeneration (lutein); or heart disease (lycopene). An effective amount of a bioflavonoid is an amount sufficient to provide a beneficial effect, such as decrease the incidence of some cancers, i.e., breast, stomach, pancreas, and lung (quercetin); or heart disease (quercetin, grape seed extract). An effective amount of α-lipoic acid is an amount sufficient to provide a beneficial effect, such as reduce the incidence of cataracts or neurologic disease. An effective amount of coenzyme Q10 is an amount sufficient to provide a beneficial effect, such as reduce the incidence of some cancers or heart disease. Such effective amounts can be determined without undue experimentation by those skilled in the art.

As used herein, “nanoparticle” means a microscopic particle with at least one dimension less than 100 nm. Liposomes are one well-known example of nanoparticles. Methods of making nanoparticles are well known in the art. Attrition is one such method. In attrition, macro- or micro-scale particles are ground in a ball mill, planetary ball mill, or other size-reducing apparatus. The resulting air particles are air classified to recover nanoparticles.

As used herein, “clustered water” or “structured water” refers to aqueous preparations known in the art and described in U.S. Pat. No. 5,247,179; U.S. Pat. No. 5,711,950; and U.S. Pat. No. 6,033,678; and the like.

As used herein, “lotions” are liquid cosmetics, often suspensions or dispersions, intended for external application to the body.

As used herein, “creams” are soft cosmetic-type preparations. Creams of the oil-in-water (O/W) type include preparations such as foundation creams, hand creams, shaving creams, and the like. Creams of the water-in-oil (W/O) type include cold creams, emollient creams, and the like. Pharmaceutically, creams are solid emulsions containing suspensions or solutions of active ingredients for external application. Generally, preparations of this type are classified as ointments. Specifically, they belong to the emulsion-type bases.

As used herein, “ointments” are semisolid preparations for external application of such consistency that may be readily applied to the skin. They should be of such composition that they soften, but not necessarily melt, when applied to the body. They serve as vehicles for the topical application of active ingredients and also function as protectives and emollients for the skin. For many years ointments were limited by definition and use to mixtures of fatty substances. Today, in addition to such oleaginous mixtures, there are ointment preparations possessing the same general consistency but entirely free of oleaginous substances. In many instances, they are emulsions of fatty or wax-like materials with comparatively high proportions of water. These emulsions may be either water-in-oil (W/O) or oil-in-water (O/W) emulsions, depending primarily on the selection of the emulsifying agent. Such semisolid emulsions are also referred to as creams. Creams and ointments containing large amounts of insoluble powders are referred to as pastes. Pastes are usually stiffer and more absorptive than creams and ointments.

The present invention advances prior art dietary supplements by providing a dietary supplement formulated with fucoidan from seaweed, such as limu moui, kombu, or mozuku. The addition of fucoidan to the dietary supplement of the present invention serves to provide significant dietary and nutritional advantages not found in prior art dietary supplements. The fucoidan-enhanced dietary supplement of the present invention provides many beneficial functions, including providing for life extension, anti-aging, and regeneration of cells and tissues, such as muscles and bones; promoting growth factors in the body; promoting high energy, vitality, and youthfulness; maintaining and strengthening the immune system, reducing allergies, inhibiting blood clotting, controlling blood sugar, preventing ulcers, reliving stomach disorders, reducing inflammation, protecting the kidneys, and detoxifying the body. Fucoidan preparations according to the present invention may also help to reduce and prevent cardiovascular disease by lowing cholesterol levels, inhibiting smooth muscle cell proliferation, and activating enzymes involved in the beta-oxidation of fatty acids.

In addition, the fucoidan-enhanced dietary supplement of the present invention fights cancerous tumors and minimizes the visible signs of both biological and environmental aging. That is, the present dietary supplements slow the aging process. assist in regenerating damaged cells and tissues, and promote growth factors in the body. Fucoidan is high in antioxidants that help to fight free radical damage to the body that may lead to cancer. Fucoidan also provides significant benefits to the skin. Fucoidan is high in antioxidants that help to fight free radical damage caused by the sun and other changing environmental conditions and elements.

Brown seaweed grows in many oceans, including off the coasts of Japan and Okinawa, Russian coastal waters, Tonga, and other places. An excellent source of fucoidan is the limu moui sea plant growing in the waters of the Tongan islands. This brown seaweed contains many vitamins, minerals, and other beneficial substances and is particularly rich in fucoidan.

Typically, the brown seaweed grows in long angel hair stems with numerous leaves. The fucoidan ingredient is found in natural compositions on the cell walls of the seaweed, providing a slippery sticky texture that protects the cell walls from the sunlight.

In one embodiment, a kombu-type or mozuku-type seaweed is harvested from the coastal waters of the Tongan islands. These seaweeds are typically manually harvested, including stems and leaves, by divers and cleaned to remove extraneous materials. The seaweed is then usually frozen in large containers and shipped to a processing plant.

In processing, the heavy outer fibers must first be broken down to provide access to the fucoidan component. If frozen, the seaweed material is first thawed, but if not frozen, then the seaweed materia is placed in a mixing vat and shredded, while being hydrolyzed with acids and water. The material can optionally be sulfonated with sulfuric acid to help in breaking down the heavy cell fibers. The mixture is also buffered with citric acid and thoroughly blended to maintain suspension. The material may also be heated at atmospheric or greater than atmospheric pressure while mixing. The resulting puree is tested and maintained at a pH of about 2 to 4 so as to remain acidic, enhancing preservative and stability characteristics.

The puree may be used in preparing dietary supplement products. Alternately, the mixture may be refrozen in small containers for later processing.

The present invention provides a dietary supplement beverage formulated with fucoidan compositions from seaweed, such as the limu moui seaweed plant. The fucoidan compositions are present in selected embodiments from about 0.5 to about 70 percent by weight of the total weight of the composition. Other ingredients may include an antioxidant, such a acai fruit and blueberry having a high oxygen radical absorbance capacity (ORAC). Such antioxidants may be present in amounts from about 0 to about 20 percent by weight. Additionally, minerals such as deep sea minerals may be present in an amount from about 0 to about 2 percent by weight, to provide important minerals.

High ORAC Nutraceutical Ingredients

Free radicals are very reactive and highly destructive compounds in the body. Free radicals are products of oxidative deterioration of such substances as polyunsaturated fat. Antioxidants convert free radical into a less reactive and nonharmful chemical form. Antioxidants that can be used in dietary supplements include β-carotene, vitamin E, vitamin C, N-acetyl cysteine, α-lipoic acid, selenium, and the like. Antioxidants having a high ORAC value are particularly desirable. Illustratively, nutraceutical antioxidants of high ORAC value that can be used in the present invention include concentrates of grape (red, black, or white), blueberry, acai fruit, raspberry, blackberry, strawberry, plum, orange, cherry, kiwi fruit, currant, elderberry, black currant, cranberry, mangosteen, noni, aronia, wolfberry, and mixtures thereof. Other high ORAC nutraceutical ingredients include proanthocyanidins, such as oligomeric proanthocyanidins, curcuminoids, and the like.

Minerals

Minerals serve a wide variety of essential physiological functions ranging from structural components of body tissues to essential components of many enzymes and other biological important molecules. Minerals are classified as micronutrients or trace elements on the basis of the amount present in the body. The seven micronutrients (calcium, potassium, sodium, magnesium, phosphorus, sulfur, and chloride) are present in the body in quantities of more than five grams. Trace elements, which include boron, copper, iron, manganese, selenium, and zinc are found in the body in quantities of less than five grams.

Micronutrient Minerals. Calcium is the mineral element believed to be most deficient in the diet in the United States. Calcium intakes in excess of 300 mg per day are difficult to achieve in the absence of milk and dairy products in the diet. This is far below the recommended dietary allowance (RDA) for calcium (1000 mg per day for adults and children ages one to ten, 1200 mg per day for adolescents and pregnant and lactating women, which equates to about four glasses of milk per day). In fact, it has been reported that the mean daily calcium intake for females over age 12 does not exceed 85 percent of the RDA. In addition, during the years of peak bone mass development (18 to 30), more than 66 percent of all U.S. women fail to consume the recommended amounts of calcium on any given day. After age 35, this percentage increases to over 75 percent.

Although the general public is not fully aware of the consequences of inadequate mineral intake over prolonged periods of time, there is considerable scientific evidence that low calcium intake is one of several contributing factors leading to osteoporosis. In addition, the dietary ratio of calcium to phosphorous (Ca:P) relates directly to bone health. A Ca to P ratio of 1:1 to 2:1 is recommended to enhance bone marrowization in humans. Such ratios are difficult to achieve absent an adequate dietary supply of milk and dairy products, or an adequate supply of calcium and other minerals for the lactose-intolerant segment of the population.

Magnesium is the second most plentiful cation of the intracellular fluids. It is essential for the activity of many enzyme systems and plays an important role with regard to neurochemical transmission and muscular excitability. Deficits are accompanied by a variety of structural and functional disturbances. The average 70-kg adult has about 2000 mEq of magnesium in his body. About 50% of this magnesium is found in bone, 45% exists as an intracellular cation, and 5% is in the extracellular fluid. About 30% of the magnesium in the skeleton represents an exchangeable pool present either within the hydration shell or on the crystal surface. Mobilization of the cation from this pool in bone is fairly rapid in children, but not in adults. The larger fraction of magnesium in bone is apparently an integral part of bone crystal.

The average adult in the United States ingests about 20 to 40 mEq of magnesium per day in an ordinary diet, and of this about one third is absorbed from the gastrointestinal tract. The evidence suggests that the bulk of the absorption occurs in the upper small bowel. Absorption is by means of an active process apparently closely related to the transport system for calcium. Ingestion of low amounts of magnesium results in increased absorption of calcium and vice versa.

Magnesium is a cofactor of all enzymes involved in phosphate transfer reactions that utilize adenosine triphosphate (ATP) and other nucleotide triphosphates as substrates. Various phosphatases and pyrophosphatases also represent enzymes from an enormous list that are influenced by this metallic ion.

Magnesium plays a vital role in the reversible association of intracellular particles and in the binding of macromolecules to subcellular organelles. For example, the binding of messenger RNA (mRNA) to ribosomes is magnesium dependent, as is the functional integrity of ribosomal subunits. Certain of the effects of magnesium on the nervous system are similar to those of calcium. An increased concentration of magnesium in the extracellular fluid causes depression of the central nervous system (CNS). Hypomagnesemia causes increased CNS irritability, disorientation, and convulsions. Magnesium also has a direct depressant effect on skeletal muscle. Abnormally low concentrations of magnesium in the extracellular fluid result in increased acetylcholine release and increased muscle excitability that can produce tetany.

Trace Elements. Boron is required by the body in trace amounts for proper metabolism of calcium, magnesium, and phosphorus. Boron helps brain function, healthy bones, and can increase alertness. Boron is also useful for people who want to build muscle. Boron is known to help prevent postmenopausal osteoporosis. Further, a relationship has been shown between a lack of boron in the diet and the chances of developing arthritis. R. E. Newnham, 46 Journal of Applied Nutrition (1994).

Chromium is an important trace element wherein the lack of sufficient chromium in the diet leads to impairment of glucose utilization, however, disturbances in protein and lipid metabolism have also been observed. Impaired glucose utilization occurs in many middle-aged and elderly human beings. In experimental studies, significant numbers of such persons have shown improvement in their glucose utilization after treatment with chromium. Chromium is transported by transferrin in the plasma and competes with iron for binding sites. Chromium as a dietary supplement may produce benefits due to its enhancement of glucose utilization and its possible facilitating the binding of insulin to insulin receptors, which increases its effects on carbohydrate and lipid metabolism. Chromium as a supplement may produce benefits in atherosclerosis, diabetes, rheumatism, and weight control.

Copper is another important trace element in the diet. The most common defect observed in copper-deficient animals is anemia. Other abnormalities include growth depression, skeletal defects, demyelination and degeneration of the nervous system, ataxia, defects in pigmentation and structure of hair or wool, reproductive failure and cardiovascular lesions, including dissecting aneurisms. Several copper-containing metalloproteins have been isolated, including tyrosinase, ascorbic acid oxidase, laccase, cytochrome oxidase, uricase, monoamine oxidase, δ-aminolevulinic acid hydrydase, and dopamine-β-hydroxylase. Copper functions in the absorption and utilization of iron, electron transport, connective tissue metabolism, phospholipid formation, purine metabolism, and development of the nervous system. Ferroxidase I (ceruloplasmin), a copper-containing enzyme, effects the oxidation of Fe(II) to Fe(III), a required step for mobilization of stored iron. A copper-containing enzyme is thought to be responsible for the oxidative deamination of the epsilon amino group of lysine to produce desmosine and isodesmosine, the cross-links of elastin. In copper-deficient animals the arterial elastin is weaker and dissecting aneurisms may occur.

Iodine is important for the production of thyroid hormones, which regulate cellular oxidation. The iodine-deficiency disease is goiter. In iodine-deficient young, growth is depressed and sexual development is delayed, the skin and hair are typically rough, and the hair becomes thin. Cretinism, feeble-mindedness, and deaf-mutism occur in a severe deficiency. There is reproductive failure in females and decreased fertility in males that lack sufficient iodine in the diet.

Iron is an essential component of several important metalloproteins. These include hemoglobin, myoglobin, and many oxidation-reduction enzymes. In iron deficiency, there may be reduced concentrations of some of the iron-containing enzymes, such as cytochrome c in liver, kidney, and skeletal muscle, and succinic dehydrogenase in the kidney and heart.

Manganese plays a role in the synthesis of GAGs, collagen, and glycoproteins, which are important constituents of cartilage and bone. Manganese is required for enzyme activity of glycosyltransferases. This family of enzymes is responsible for linking sugars together into GAGs, adding sugars to other glycoproteins, adding sulfate to aminosugars, converting sugars to other modified sugars, and adding sugars to lipids. These functions are manifested as GAG synthesis (hyaluronic acid, chondroitin sulfate, karatan sulfate, heparin sulfate, and dermatin sulfate, among others), collagen synthesis, and function of many other glycoproteins and glycolipids. GAGs and collagen are chief structural elements for all connective tissues. Their synthesis is essential for proper maintenance and repair of connective tissues.

Manganese deficiencies in humans and animals lead to abnormal bone growth, swollen and enlarged joints, and slipped tendons. In humans, manganese deficiencies are associated with bone loss, arthritis, and impaired glucose utilization. Levels of all GAGs are decreased in connective tissues during manganese deficiencies, with chondroitin sulfates being most depleted. Manganese-deficient organisms quickly normalize GAG and collagen synthesis when manganese is provided.

Manganese is also required for activity of manganese superoxide dismutase (MnSOD), which is present only in mitochondria. Manganese deficiency decreases the activity of MnSOD and may lead to mitochondrial dysfunction, manifested as decreased cellular functions. Manganese is required for the conversion of mevalonic acid to squalene. Pyruvate carboxylase is a manganese metalloenzyme, repressible by insulin, important in the citric acid cycle for the oxidation of carbohydrates, lipids, and proteins, as well as in the synthesis of glucose and lipids.

Molybdenum is an essential mineral found in highest concentrations in the liver, kidneys, skin, and bones. This mineral is required by the body to properly metabolize nitrogen. It is also a vital component of the enzyme xanthine oxidase, which is required to convert purines to uric acid, a normal byproduct of metabolism. Molybdenum also supports the body's storage of iron and other cellular functions such as growth. A deficiency of molybdenum is associated with mouth and gum disorders and cancer. A diet high in refined and processed foods can lead to a deficiency of molybdenum, resulting in anemia, loss of appetite and weight, and stunted growth in animals. While these deficiencies have not been observed directly in humans, it is known that a molybdenum deficiency can lead to impotence in older males.

Selenium is an essential trace element that functions as a component of enzymes involved in protection against antioxidants and thyroid hormone metabolism. In several intra- and extra-cellular glutathione peroxidases and iodothyronine 5′-deiodinases, selenium is located at the active centers as the selenoamino acid, selenocysteine (SeCYS). At least two other proteins of unknown function also contain SeCYS. Although SeCYS is an important dietary form, it is not directly incorporated into these specific selenium-proteins; instead, a co-translational process yields tRNA-bound SeCYS. In contrast, selenium as seleno-methionine is incorporated non-specifically into many proteins, as it competes with methionine in general protein synthesis. Therefore, tissues often contain both specific, as well as the nonspecific, selenium-containing proteins when both SeCYS and selenomethionine are consumed, as found in many foods. Selenium is a major antioxidant nutrient and is involved in protecting cell membranes and preventing free radical generation, thereby decreasing the risk of cancer and disease of the heart and blood vessels. Medical surveys show that increased selenium intake decreases the risk of breast, colon, lung and prostate cancer. Selenium also preserves tissue elasticity; slows down the aging and hardening of tissues through oxidation; and helps in the treatment and prevention of dandruff. Recent research has shown antitumorigenic effects of high levels of selenium in the diets of several animal models.

Vanadium is an essential nutrient beneficial for thyroid hormone metabolism. The daily requirement necessary to prevent a deficiency is about 10 to 20 micrograms a day. Vanadium deficiency can lead to slow growth, defective bones, and altered lipid metabolism. Vanadium exerts an insulin-like effect in some respects, and there has been a considerable amount of research on vanadium and diabetes. In insulin dependent diabetics, vanadium has been found to reduce the amount of insulin required to manage the disease, and in non-insulin dependent diabetics, vanadium has been known to control the condition altogether. Research has shown that supplementation with vanadium leads to an increase in glucose transport into cells, which suggests that vanadium supplementation of the diet improves glucose metabolism and may aid in preventing diabetes.

Zinc is known to occur in many important metalloenzymes. These include carbonic anhydrase, carboxypeptidases A and B, alcohol dehydrogenase, glutamic dehydrogenase, D-glyceraldehyde-3-phosphate dehydrogenase, lactic dehydrogenase, malic dehydrogenase, alkaline phosphatase, and aldolase. Impaired synthesis of nucleic acids and proteins has been observed in zinc deficiency. There is also evidence that zinc may be involved in the secretion of insulin and in the function of the hormone.

According to the present invention, minerals can be provided as inorganic compounds, such as chlorides, sulfates, and the like. In addition, some minerals can be provided in more bioavailable forms, such as amino acid chelates, which are well known in the art. U.S. Pat. No. 5,292,538. Examples of minerals that can be provided as amino acid chelates include calcium, magnesium, manganese, zinc, iron, boron, copper, molybdenum, and chromium. Still further, minerals can be provided as deep sea minerals.

Carotenoids

Carotenoids are a family of hundreds of plant pigments found in fruits and vegetables that are red, orange, and deep yellow in color, and also in some dark green leafy vegetables. See USDA-NCC Carotenoid Database for U.S. Foods (1998). Carotenoids are the precursors of most of the vitamin A found in animals. At least 10 different carotenoids exhibit provitamin A activity, including α- and β-carotenes and cryptoxanthin. As precursors of vitamin A, carotenoids exhibit an effect on vision, but carotenoids are known to have other beneficial effects in the diet, as well. For example, carotenoids are also known for their antioxidant activity in helping protect the body from free radical damage.

Volumes of research reveal that two carotenoids—lutein and zeaxanthin—are found in great concentrations in the macula of the eye. This research also indicates that maintaining high levels of these two carotenoids, especially lutein, may help diminish the effects of age-related macular degeneration, the leading cause of blindness in those over 65 years of age. Lutein acts as an antioxidant, protecting cells against the damaging effects of free radicals. As with the other carotenoids, lutein is not made in the body and, therefore, must be obtained from food or dietary supplements.

At one time researchers believed all antioxidants served the same purpose. Now there is growing evidence that individual antioxidants may be used by the body for specific purposes. Researchers believe that lutein is deposited into areas of the body most prone to free radical damage. One major example is the macula, a tiny portion of the retina. Research indicates that because of its antioxidant properties, lutein consumption may play a role in maintaining the health of the eyes, heart and skin as well as the breasts and cervix in women. In addition, scientists are studying lutein's possible role in age-related macular degeneration, cataracts, heart disease, and immune system health. Studies have also shown that lutein is associated with a reduction in lung, breast, and cervical cancer. In the vascular system, lutein is found in high-density lipoprotein (“HDL”) or “good” cholesterol and may prevent low-density lipoprotein (“LDL”) or “bad” cholesterol from oxidizing, which sets the cascade for heart disease.

Besides being a precursor of vitamin A, β-carotene is thought to be effective in helping to protect against some diseases, such as cancer, heart disease, and stroke.

Lycopene is an open-chain unsaturated carotenoid that imparts red color to tomatoes, guava, rosehip, watermelon, and pink grapefruit. Lycopene is a proven anti-oxidant that may lower the risk of certain diseases including cancer and heart disease. In the body, lycopene is deposited in the liver, lungs, prostate gland, colon, and skin. Its concentration in body tissues tends to be higher than all other carotenoids. Epidemiological studies have shown that high intake of lycopene-containing vegetables is inversely associated with the incidence of certain types of cancer. For example, habitual intake of tomato products has been found to decrease the risk of cancer of the digestive tract among Italians. In one six-year study by Harvard Medical School and Harvard School of Public Health, the diets of more than 47,000 men were studied. Of 46 fruits and vegetables evaluated, only the tomato products (which contain large quantities of lycopene) showed a measurable relationship to reduce prostate cancer risk. As consumption of tomato products increased, levels of lycopene in the blood increased, and the risk for prostate cancer decreased. Ongoing research suggests that lycopene can reduce the risk of macular degenerative disease, serum lipid oxidation, and cancers of the lung, bladder, cervix and skin. Studies are underway to investigate other potential benefits of lycopene including lycopene's potential in the fight against cancers of the digestive tract, breast, and prostate. W. Stahl & H. Sies, Lycopene: a biologically important carotenoid for humans? 336 Arch. Biochem. Biophys. 1-9 (1996); H. Gerster, The potential role of lycopene for human health, 16 J. Amer. Coll. Nutr. 109-126 (1997).

Flavonoids

Flavonoids (also called bioflavonoids) are natural botanical pigments that provide protection from free-radical damage, among other functions. Bioflavonoids provide protection from damaging free radicals and are believed to reduce the risk of cancer and heart disease, decrease allergy and arthritis symptoms, promote vitamin C activity, improve the strength of blood vessels, block the progression of cataracts and macular degeneration, treat menopausal hot flashes, and other ailments. Flavonoids occur in most fruits and vegetables. It is believed that flavonoids act by inhibiting hormones, such as estrogen, that may trigger hormone-dependent malignancies like cancers of the breast, endometrium, ovary, and prostate. Studies show that quercetin, a flavonoid found in citrus fruits, can block the spread of cancer cells in the stomach. Flavonoids also stabilize mast cells, a type of immune cell that releases inflammatory compounds, like histamine, when facing foreign microorganisms. Histamine and other inflammatory substances are involved in allergic reactions. Mast cells are large cells present in connective tissue. Flavonoids fortify and repair connective tissue by promoting the synthesis of collagen. Collagen is a remarkably strong protein of the connective tissue that “glues” the cells together. Flavonoids are believed to benefit connective tissue and reduce inflammation.

Citrus bioflavonoids include isoquercetin, quercetin, hesperidin, rutin, naringen, naringenin, and limonene. Isoquercetin is a common flavonoid found in onions, apples, Arnica species, Gossypium arboreum, Ginko biloba, Ricinus communis, Ocimum basilicum, Salix acutifolia, and Narcissus pseudonarcissus. Rich dietary sources of quercetin are onions, apples, kale, sweet cherries, grapes, red cabbage, and green beans. Hesperidin is found in the rinds of oranges and lemons. It helps strengthen capillary walls in conjunction with vitamin C. Naringen is found in grapefruit and is responsible for most of grapefruit's bitter taste. Limonene, a flavonoid available in citrus fruits, promotes the production of enzymes that help destroy possible carcinogens (cancer-causing agents). Other bioflavonoids include: isoflavones, proanthocyanidins, anthocyanidins, ellagic acid, catechin, and tannin.

Isoquercetin shares the same aglycone with rutin and quercitrin: quercetin. It has been shown that quercetin-containing glycosides liberate quercetin in the intestinal tract. Therefore, it is justified to assume that all the pharmacological properties of quercetin are also shared by isoquercetin and rutin when administered orally. Recent investigation demonstrated a rapid absorption of isoquercetin and quercetin-glucosides by the sodium-dependent glucose transport pathway in the small intestine. Due to superior bioavailability, the health effects of isoquercetin are increased compared to other flavonoids. Isoquercetin is known to have anti-inflammatory activity without adverse effects on the gastrointestinal tract, such as those caused by non-steroidal anti-inflammatory drugs (NSAIDs). Isoquercetin further exhibits beneficial effects as an antioxidant, antihypertensive, anticarcinogenic, antimicrobial, and analgesic agent.

Quercetin is a bioflavonoid and a natural reverse transcriptase blocker commonly found in red apples and red onions. Quercetin has been shown to have antiviral activity against HIV, herpes simplex, and the respiratory syncytial virus. T. N. Kaul et al., Antiviral effects of flavonoids on human viruses, 15 J. Med. Virol. 71-79 (1985); R. Vrijsen et al., Antiviral activity of flavones and potentiation by ascorbate, 69 J. Gen. Virol. 1749-1751 (1988).

Grape seed extract is another source of bioflavonoids. Grape seed extract has been known to exhibit the following benefits: anti-inflammatory, antihistamine, antiallergenic, antioxidant (free radical scavenger), helps skin to remain young looking, improves circulation, promotes healing, restores collagen, strengthens weak blood vessels, and improves tissue elasticity. Some known applications include treatment of arthritis, allergies, hardening of arteries, ulcers, and skin problems.

Isoflavones are another group of phytochemicals that provide beneficial effects when provided as supplements to the diet. Isoflavones are also known as phytoestrogens (plant estrogens) and are one-hundredth to one-thousandth as potent as human estrogen. Although they are weak estrogens, researchers are finding that they can help offset the drop in estrogen that occurs naturally at menopause. Isoflavones act like hormone replacement therapy (HRT), easing hot flashes. The main dietary sources of isoflavones are soybeans and soy foods, although some other legumes also contain small amounts. It's not clear how much soy actually is needed to get the most health benefit. Studies have shown that it may take as little as 20 grams of soy protein (about half an ounce), or about 2 cups of soy milk, or 2 ounces of tofu daily to help lessen symptoms.

Research also is underway to identify the roles isoflavones may play in protection from breast and prostate cancers. Isoflavones and soy protein also may prevent bone loss that leads to osteoporosis. Also, soy protein is being investigated for its lipid lowering effects. The most researched isoflavones are genistein, daidzein and glycitein. Data on the isoflavone content of foods is limited, however, the United States Department of Agriculture (USDA)—Iowa State University Isoflavone Database lists some common foods and their isoflavone content.

Aminosugars and Glycosaminoglycans (Cartilage Protectors)

The connective tissues are constantly subjected to stresses and strains from mechanical forces that can result in afflictions, such as arthritis, joint inflammation, and stiffness. Such afflictions are especially acute in joints, such as the neck, back, arms, hips, knees, ankles, and feet. Indeed, connective tissue afflictions are quite common, presently affecting millions of Americans. Further, such afflictions can be not only painful, but can also be debilitating.

The connective tissues are naturally equipped to repair themselves by manufacturing and remodeling prodigious amounts of collagen and proteoglycans (the major components of connective tissues). This ongoing process is placed under stress when an injury occurs to connective tissues. In such cases, the production of connective tissue (along with collagen and proteoglycans) can double or triple over normal amounts, thereby increasing the demand for the building blocks of both collagens and proteoglycans. The building blocks for collagen are amino acids. Proteoglycans are large and complex macromolecules comprises mainly of long chains of modified sugars called glycosaminoglycans (GAGs) or mucopolysaccharides. Proteoglycans provide the framework for collagen to follow. They also hold water to give the connective tissues (especially cartilage) flexibility, resiliency, and resistance to compression. In the production of proteoglycans, the rate-limiting step is the conversion of glucose to glucosamine for the production of GAGs. Glucosamine, an aminosugar, is the key precursor to all the various modified sugars found in GAGs-glucosamine sulfate, galactosamine, N-acetylglucosamine, etc. Glucosamine also makes up 50% of hyaluronic acid, the backbone of proteoglycans, on which other GAGs, like chondroitin sulfates are added. The GAGs are then used to build proteoglycans and, eventually, connective tissue. Once glucosamine is formed, there is no turning away from the synthesis of GAGs and collagen.

The composition of the present invention preferably includes an aminosugar, such as glucosamine (preferably in a salt form) and a GAG, such as chondroitin (preferably in a salt form). The aminosugar, glucosamine, provides the primary substrate for both collagen and proteoglycan synthesis. In fact, glucosamine is the preferred substrate for proteoglycan synthesis, including chondroitin sulfates and hyaluronic acid. The glucosamine is, preferably, in a salt form so as to facilitate its delivery and uptake. The preferred salt forms are glucosamine hydrochloride and glucosamine sulfate. N-acetylglucosamine is another preferred form of glucosamine. It should be noted that, in the case of glucosamine sulfate, the sulfate may be available for later use in catalyzing the conversion of glucosamine to GAGs. The unsulfated form is desired for the production of hyaluronic acid.

Glucosamine has been shown to be rapidly and almost completely absorbed into humans after oral administration. A significant portion of the ingested glucosamine localizes to cartilage and joint tissues, where it remains for long periods of time. This indicates that oral administration of glucosamine reaches connective tissues, where glucosamine is incorporated into newly-synthesized connective tissue.

Chondroitin sulfate is a glycosaminoglycan that provides a further substrate for synthesis of proteoglycan. Once again, the provision of chondroitin in its salt, especially sulfate, form facilitates its delivery and uptake by humans. Also, the sulfate is available for sulfation of the GAGs.

Chondroitin sulfate not only provides additional organic sulfur for incorporation into cartilage, but it also has a synergistic effect with glucosamine, since its structure provides galactosamine, which is synthesized by a different pathway than glucosamine. Karzel et al., 5 Pharmacology 337-3435 (1971). In addition, chondroitin sulfate has been shown to have cardiovascular health benefits, Morrison et al., Coronary Hearth Disease and the Mucopolysaccharides 109-127 (1973), and also helps prevent degradation or breakdown of cartilage.

Hyaluronic acid (HA) is a large glycoaminoglycan that contains repeating disaccharide units of N-acetyl glucosamine and glucuronic acid. It occurs in the extracellular matrix and on the cell surface. It has been shown to promote cell mobility, adhesion, and proliferation, and it has an important role in such processes as morphogenesis, wound repair, inflammation, and metastasis.

Amino Acids

The nutritional value of proteins in the human diet involves recognition of the quality as well as the quantity of the protein. Humans do not have the ability to synthesize all the amino acids required for normal good health. Those that are required to be supplied by the diet are called essential amino acids and include leucine, isoleucine, lysine, methionine, phenylalanine, threonine, tryptophan, and valine. In general, it is recommended that an adult should take in the daily diet 10 g of protein per kg of body weight. Children require about two to three times this amount. Of course, this assumes that the protein in the diet has an adequate amount of all essential and nonessential amino acids. Proteins found in eggs, beef, and milk are considered to have the best nutritional value.

Adequate protein nutrition requires the intake of sufficient protein to meet daily requirements. This protein must be of the necessary quality, i.e., supply the essential amino acids. Protein deficiency thus may be caused by a reduced intake or the use of low-quality protein. Obviously, the actual intake of protein may be influenced by factors such as high excretion in conditions of kidney damage or blood loss, or an increased requirement associated with thyrotoxicosis or high fever. Symptoms of deficiency include loss of weight, nutritional edema, and skin changes and are associated with such conditions as nephrosis, sprue, and colitis. Deficiency may result also in a reduced resistance to infection, since an adequate protein intake is necessary for the formation of phagocytes, leukocytes, and antibodies. Stress, such as brought on by accidental or surgical trauma, pregnancy, and lactation may also cause a deficiency of amino acids, and greater intakes of protein are required in these conditions.

Arginine is useful in enhancing the immune system, and it increases the size and activity of the thymus gland, which is responsible for manufacturing T lymphocytes, which are part of the immune system. Arginine is also important in liver health in that it assists in neutralizing ammonia. It is also involved in the skin and connective tissues, thus it is important in healing and repair of tissues, as well as the formation of collagen and building of new bone and tendons.

Cysteine is critical to the metabolism of a number of essential biochemicals, including coenzyme A, heparin, biotin, lipoic acid, and glutathione. Cysteine, which may be supplied as N-acetylcysteine, helps in strengthening the protective lining of the stomach and intestines. It is a constituent of the antioxidant, glutathione.

Glycine is required for building protein in the body and for synthesis of nucleic acids. Glycine has been found to be useful in aiding the absorption of calcium in the body. It is important for prostate health, and it is used by the nervous system as an inhibitory neurotransmitter, which is important for preventing epileptic seizures and for the treatment of bipolar disorder and hyperactivity.

Histidine is needed for growth and for the repair of tissue, as well as the maintenance of the myelin sheath, which acts as a protector for nerve cells. Histidine is also required for the manufacture of both red and white blood cells, and it helps to protect the body from damage caused by radiation and in removing heavy metals from the body. In the stomach, histidine is also helpful in producing gastric juices.

Isoleucine, together with the other two branched-chain amino acids, promotes muscle recovery after physical exercise. It is also needed for the formation of hemoglobin and for assisting with regulation of blood sugar levels and energy levels. It is also involved in blood clot formation.

Leucine helps with the regulation of blood-sugar levels, the growth and repair of muscle tissue, growth hormone production, wound healing, and energy regulation.

Lysine is required for growth and bone development in children, assists in calcium absorption, and assists in maintaining the correct nitrogen balance in the body and maintaining lean body mass. Further, lysine is needed to produce antibodies, hormones, enzymes, and collagen and to repair tissues.

Methionine assists in the breakdown of fats and thereby prevents the build-up of fat in the arteries. It also assists with proper functioning of the digestive system and for removing heavy metals from the body, since it can be converted to cysteine, a precursor to glutathione, which is of prime importance in detoxifying the liver. Methionine is also a great antioxidant, since the sulfur supplied in methionine inactivates free radicals. Methionine may also be used to treat depression, arthritis pain, and chronic liver disease. It is one of the three amino acids needed by the body to manufacture creatine, a compound essential for energy production and muscle building.

Phenylalanine is used for elevating mood, since it is closely involved with the nervous system. It also helps with memory and learning and has been used as an appetite suppressant.

Threonine is required to help maintain proper protein balance in the body, as well as assisting in formation of collagen and elastin in the skin. It is also involved in liver functioning (including fighting fatty liver), lipotropic functions—along with aspartic acid and methionine, and assisting in the immune system by helping the production of antibodies and promoting thymus growth and activity.

Tryptophan is required for the production of the vitamin, niacin. It is also used by the body to produce serotonin, a neurotransmitter that is important for normal nerve and brain function. Serotonin is important in sleep, stabilizing emotional moods, pain control, fighting inflammation, and maintaining intestinal peristalsis. It is also important in controlling hyperactivity in children, assisting in alleviating stress, helping with weight loss, and reducing appetite.

Valine is needed for and has a stimulating effect on muscle metabolism. It is also needed for repair and growth of tissue and maintaining the nitrogen balance in the body.

Other Nutrients

Alpha-lipoic acid (technically known as DL-alpha lipoic acid) is a powerful antioxidant being researched for unique properties that may provide both preventive and therapeutic benefits in numerous conditions and diseases including diabetes, heart disease, and even possibly HIV infection. Lipoic acid and its reduced form, DHLA, show the ability to directly quench a variety of reactive oxygen species, inhibit reactive oxygen generators, and spare and regenerate other antioxidants. Lipoic acid not only protects the nervous system, but is also involved in regenerating nerves. It is also being studied in the treatment of Parkinson's disease and Alzheimer's disease. Lipoic acid is best known for its ability to help regenerate damaged liver tissue when nothing else will. Lipoic acid is marketed in Germany for treating diabetic neuropathy. It also has an essential role in mitochondrial dehydrogenase reactions.

Coenzyme Q10 is an essential electron and proton carrier that functions in the production of biochemical energy in aerobic organisms. Coenzyme Q10 is found in every cell in the body, thus its other name, ubiquinone (from the word ubiquitous and the coenzyme quinone). The structure of coenzyme Q10 consists of a quinone ring attached to an isoprene side chain. Because the body must have energy available to perform even the simplest operation, coenzyme Q10 is considered essential for the body's cells, tissues, and organs. Coenzyme Q10 also has antioxidant and membrane stabilizing properties that serve to prevent the cellular damage that results from normal metabolic processes. Even though the body has the ability to produce coenzyme Q10, deficiencies have been reported in a range of clinical conditions. Supplementation of the coenzyme helps guard against a possible deficiency. Aging is considered one reason for a deficiency, since the liver loses its ability to synthesize coenzyme Q10 as one gets older. Besides aging, poor eating habits, stress, and infection affect the body's ability to provide adequate amounts of coenzyme Q10. Known results of using coenzyme Q10 as an oral supplement are energy increase, improvement of heart function, prevention and cure of gum disease, a boost to the immune system, and possible life extension. AIDS is a primary target for research on coenzyme Q10 because of its immense benefits to the immune system. Further, coenzyme Q10 has also been reported to provide a salutary effect in the treatment of breast cancer.

Vitamins

Vitamins are organic compounds that are required for the normal growth and maintenance of life of animals, including man, who are generally unable to synthesize these compounds by anabolic processes that are independent of environment other than air, and which compounds are effective in small amounts, do not furnish energy, and are not utilized as building units for the structure of the organism, but are essential for the transformation of energy and for the regulation of the metabolism of structural units. Vitamins or their precursors are found in plants, and thus plant tissues are the sources for the animal kingdom of these protective nutritional factors. In addition to carbohydrates, fats, proteins, mineral salts, and water, it is essential that the food of man and animals contain small amounts of these vitamins. If any one of at least 13 of these compounds is lacking in the diet, a breakdown of the normal metabolic processes occurs, which results in a reduced rate or complete lack of growth in children and in symptoms of malnutrition that are classified as deficiency diseases.

The functions of vitamins generally fall into two categories, the maintenance of normal structure and the maintenance of normal metabolic functions. For example, vitamin A is essential for the maintenance of normal epithelial tissue, and vitamin D functions in the absorption of normal bone salts for the formation and growth of a sound bony structure. Certain vitamins, such as thiamine, riboflavin, pantothenic acid, and niacin, are known to be essential constituents of the respiratory enzymes that are required in the utilization of energy from oxidative catabolism of sugars and fats.

It is convenient to divide vitamins into two groups, the water-soluble vitamins and the fat-soluble vitamins. The water-soluble vitamins include ascorbic acid and the B group of vitamins, which consists of some 10 or more well-defined compounds. The fat-soluble vitamins include vitamins A, D, E, and K, since they can be extracted with organic solvents and are found in the fat fractions of animal tissues. For brief reviews of vitamins in general and specific vitamins, see Remington's Pharmaceutical Sciences.

Fat-soluble vitamins. Vitamin A is essential for the maintenance of normal tissue structure and for other important physiologic functions such as vision and reproduction. The source of most of the vitamin A in animals is the carotenoid pigments, i.e. the yellow-colored compounds in all chlorophyll-containing plants. At least 10 different carotenoids exhibit provitamin A activity. For example, α- and β-carotene and cryptoxanthin (found in yellow corn) are important in animal nutrition, β-carotene being the most important. Theoretically, one molecule of β-carotene should yield two molecules of vitamin A. The availability of carotene in foods as sources of vitamin A for humans, however, is low and extremely variable. The conversion of the provitamin to vitamin A occurs primarily in the walls of the small intestine and perhaps to a lesser degree in the liver. Like vitamin A, the carotenes are soluble in organic solvents.

Of the known functions of vitamin A in the body, its role in vision is established best. The retina of man contains two distinct photoreceptor systems. The rods, which are the structural components of one system, are especially sensitive to light of low intensity. A specific vitamin A aldehyde is essential for the formation of rhodopsin, the high molecular weight glycoprotein part of the visual pigment within the rods, and the normal functioning of the retina. By virtue of this relation in the visual process, vitamin A alcohol has been named retinol, and the aldehyde form is named retinal. A vitamin-A deficient person has impaired dark adaptation (“night-blindness”).

Vitamin A also aids in the differentiation of cells of the skin (lining the outside of the body) and mucous membranes (linings inside of the body); helps the body fight off infection and sustain the immune system; and, supports growth and remodeling of bone. In addition, dietary vitamin A, in the form of its precursor β-carotene (an antioxidant), may help reduce risk for certain cancers.

Vitamin D is the vitamin effective in promoting calcification of the bony structures of man and animals. It is sometimes known as the “sunshine” vitamin because it is formed by the action of the sun's ultraviolet rays on precursor sterols in the skin. Vitamin D aids in the absorption of calcium from the intestinal tract and the resorption of phosphate in the renal tubule. Vitamin D is necessary for normal growth in children, probably having a direct effect on the osteoblast cells, which influence calcification of cartilage in the growing areas of the bone. A deficiency of vitamin D leads to inadequate absorption of calcium from the intestinal tract and retention of phosphorus in the kidney and thus to faulty mineralization of bone structures. Vitamin D also maintains a stable nervous system and normal heart action.

Vitamin E is a group of compounds (tocol and tocotrienol derivatives) that exhibit qualitatively the biological activity of α-tocopherol. Biological activity associated with the vitamin nature of the group is exhibited by four major compounds: α-, β-, γ-, and δ-tocopherol, each of which can exist in various stereoisomeric forms. The tocopherols act as antioxidants, δ-tocopherol having the greatest antioxidant power. The most critical function of vitamin E occurs in the membranous parts of the cells. Vitamin E interdigitates with phospholipids, cholesterol, and triglycerides, the three main structural elements of the membranes. Since vitamin E is an antioxidant, a favored reaction is with the very reactive and highly destructive compounds called free radicals. Free radicals are products of oxidative deterioration of such substances as polyunsaturated fat. Vitamin E converts the free radical into a less reactive and nonharmful form. Vitamin E also supplies oxygen to the blood, which is then carried to the heart and other organs; thus alleviating fatigue; aids in bringing nourishment to cells; strengthens the capillary walls and prevents the red blood cells from destructive poisons; prevents and dissolves blood clots; and has also been used in helping prevent sterility, muscular dystrophy, calcium deposits in blood walls, and heart conditions.

Vitamin K is a group of substances of which the primary activity that makes the vitamin essential in human metabolism is its involvement in the blood-clotting system through synthesis of prothrombin and other clotting factors. Vitamin K contributes to biosynthesis of bone protein, and is necessary for the formation of prothrombinogen and other blood clotting factors in the liver. During clotting, circulating prothrombin is required for the production of thrombin. In turn, thrombin converts fibrinogen to fibrin, the network of which constitutes the clot. It is obvious from this description that interference with formation of prothrombin will reduce the clotting tendency of blood. In a deficiency of the vitamin, a condition of hypoprothrombinemia occurs, and blood-clotting time may be greatly, or even indefinitely, prolonged. Internal or external hemorrhages may ensue either spontaneously or following injury or surgery.

Water-soluble vitamins. Except for ascorbic acid, all of the vitamins in this category belong the B-group of vitamins. Some still retain their original individual designations, such as B-1, B-6, and B-12, whereas comparable names for other vitamins have become obsolete.

Vitamin C, or ascorbic acid, is known to be essential for the formation of intercellular collagen. Symptoms of scurvy, due to vitamin C deficiency, include bleeding gums, easy bruising and a tendency toward bone fractures. All these symptoms are a result of the requirement for vitamin C in the development of the ground substance between our cells. This ground substance, primarily collagen, is the cement that gives our tissues form and substance. Collagens are principal components of tendons, ligaments, skin, bone, teeth, cartilage, heart valves, intervertebral discs, cornea, eye lens, in addition to the ground substance between cells. Some collagen forms in the absence of ascorbic acid, but the fibers are abnormal, resulting in skin lesions and blood vessel fragility, characteristics of scurvy. In scorbutic tissues the amorphous ground substance and the fibroblasts in the area between the cells appear normal but without the matrix of collagen fibers. These bundles of collagenous material appear within a few hours after administration of ascorbic acid. This points to the relationship of the vitamin in maintenance of tooth structures, matrix of bone, and the walls of capillaries. Vitamin C is essential for the healing of bone fractures. Such fractures heal slowly in a patient deficient in vitamin C. This is true also of wound healing.

Vitamin C is also an antioxidant. Oxygen is a highly reactive element, and the process of reacting with certain chemicals is termed oxidation. Oxidation is not always bad. For example, the iron in hemoglobin oxidizes to carry oxygen to all the cells of the body. But much oxidation is damaging, accelerating aging and contributing to tissue and organ damage. Oxidation is also a contributor to heart disease (low density lipoprotein (LDL) oxidation has been linked to atherosclerosis) and cancer. As research continues, the more free-radical damage appears to contribute to chronic conditions and the more antioxidant nutrition supplementation is realized to be is essential. Vitamin C is the most effective water-soluble antioxidant in human plasma. Vitamin C is also a requirement for the proper functioning of the immune system. It is involved in white blood cell production, T-cells, and macrophages.

Biotin functions in synthesis and breakdown of fatty acids and amino acids through aiding the addition and removal of carbon dioxide to or from active compounds. It similarly acts in catalyzing deamination of amino acids and in oleic acid synthesis. Biotin is also an essential component of enzymes and aids in the utilization of protein and certain other vitamins, such as folic acid, pantothenic acid, and vitamin B-12.

Folic acid or folacin is one of the important hematopoietic agents necessary for proper regeneration of blood-forming elements and their functioning. Folic acid is also involved as a coenzyme in intermediary metabolic reactions in which one-carbon units are transferred. These reactions are important in interconversions of various amino acids and in purine and pyrimidine synthesis. The biosynthesis of purines and pyrimidines is ultimately linked with that of nucleotides and ribo- and deoxyribo-nucleic acids, functional elements in all cells.

Niacin (nicotinic acid) and niacinamide (nicotinamide) have identical properties as vitamins. In the body niacin is converted to niacinamide, which is an essential constituent of coenzymes I and II that occur in a wide variety of enzyme systems involved in anaerobic oxidation of carbohydrates. The coenzyme serves as a hydrogen acceptor in the oxidation of the substrate. These enzymes are present in all living cells and take part in many reactions of biological oxidation. Nicotinamide-adenine dinucleotide (NAD) and nicotinamide-adenine dinucleotide phosphate (NADP) are coenzymes synthesized in the body that take part in the metabolism of all living cells. Since they are of such widespread and vital importance, it is not difficult to see why serious disturbance of metabolic processes occurs when the supply of niacin to the cell is interrupted. Niacin is readily absorbed from the intestinal tract, and large doses may be given orally or parenterally with equal effect. Further, niacin improves circulation and reduces the cholesterol level in the blood; maintains the nervous system; helps metabolize protein, sugar & fat; reduces high blood pressure; increases energy through proper utilization of food; prevents pellagra; and helps maintain a healthy skin, tongue, and digestive system.

Pantothenic acid is of the highest biological importance because of its incorporation into Coenzyme A (CoA), which is involved in many vital enzymatic reactions transferring a two-carbon compound (the acetyl group) in intermediary metabolism. It is involved in the release of energy from carbohydrate and protein, in the degradation and metabolism of fatty acids, and in the synthesis of such compounds as sterols and steroid hormones, porphyrins, and acetyl-choline. Pantothenic acid also participates in the utilization of vitamins; improves the body's resistance to stress; helps in cell building & the development of the central nervous system; helps the adrenal glands, and fights infections by participating in building of antibodies.

Pyridoxine (vitamin B-6) does not denote a single substance, but is rather a collective term for a group of naturally occurring pyridines that are metabolically and functionally interrelated: namely, pyridoxine, pyridoxal, and pyridoxamine. They are interconvertible in vivo in their phosphorylated form. Vitamin B-6 in the form of pyridoxal phosphate or pyridoxamine phosphate functions in carbohydrate, fat, and protein metabolism. Its major functions are most closely related to protein and amino acid metabolism. The vitamin is a part of the molecular configuration of many enzymes (a coenzyme), notably glycogen phosphorylase, various transaminases, decarboxylases, and deaminases. The latter three are essential for the anabolism and catabolism of proteins. Pyridoxine is also aids in fat and carbohydrate metabolism; aids in the formation of antibodies; maintains the central nervous system; aids in the removal of excess fluid of premenstrual women; promotes healthy skin; reduces muscle spasms, leg cramps, hand numbness, nausea and stiffness of hands; and helps maintain a proper balance of sodium and phosphorous in the body.

Riboflavin is another B vitamin, which plays its physiological role as the prosthetic group of a number of enzyme systems that are involved in the oxidation of carbohydrates and amino acids. It functions in combination with a specific protein either as a mononucleotide containing phosphoric acid (FMN), or as a dinucleotide combined through phosphoric acid with adenine (FAD). The specificity of each of the enzymes is determined by the protein in the complex. By a process of oxidation-reduction, riboflavin in the system either gains or loses hydrogen. The substrate, either carbohydrate or amino acid, may be oxidized by a removal of hydrogen. The first hydrogen acceptor in the chain of events is NAD or NADP, the di- or tri-nucleotide containing nicotinic acid and adenine. The oxidized riboflavin system then serves as hydrogen acceptor for the coenzyme system and in turn is oxidized by the cytochrome system. The hydrogen is finally passed on to the oxygen to complete the oxidative cycle. A number of flavoprotein enzymes have been identified, each of which is specific for a given substrate. Riboflavin also aids in the formation of antibodies and red blood cells; maintains cell respiration; necessary for the maintenance of good vision, skin, nails and hair; alleviates eye fatigue; and promotes general health.

Thiamine or thiamin is a generic term applied to all substances possessing vitamin B-1 activity, regardless of the anion attached to the molecule. The cationic portion of the molecule is made up of a substituted pyrimidine ring connected by a methylene bridge to the nitrogen of a substituted thiazole ring. In a phosphorylated form, thiamine serves as the prosthetic group of enzyme systems that are concerned with the decarboxylation of α-ketoacids. Some decarboxylation reactions are reversible, so that synthesis (condensation) may be achieved. Thus, thiamine is also important to the biosynthesis of keto-acids. It is involved in transketolase reactions. Thiamine is readily absorbed in aqueous solution from both the small and large intestine, and is then carried to the liver by the portal circulation. In the liver, as well as in all living cells, it normally combines with phosphate to form cocarboxylase. It may be stored in the liver in this form or it may combine further with manganese and specific proteins to become active enzymes known as carboxylases. Thiamine also plays a key role in the body's metabolic cycle for generating energy; aids in the digestion of carbohydrates; is essential for the normal functioning of the nervous system, muscles & heart; stabilizes the appetite; and promotes growth & good muscle tone.

Vitamin B-12 or cyanocobalamin is essential for the functioning of all cells, but particularly for cells of the bone marrow, the nervous system, and the gastrointestinal tract. It appears to facilitate reduction reactions and participate in the transfer of methyl groups. Its chief importance seems to be, together with folic acid, in the anabolism of DNA in all cells. It is a requisite for normal blood formation, and certain macrocystic anemias respond to its administration. Vitamin B-12 is also necessary for carbohydrate, fat, and protein metabolism; maintains a healthy nervous system; promotes growth in children; increases energy; and is needed for calcium absorption.

Botanical Ingredients

Mangosteen is a tree that is fairly widespread in Southeast Asia and is known for its medicinal properties. The fruit hulls have been used in folk medicine for the treatment of skin infections, wounds, and diarrhea in Southeast Asia.

Turmeric has been used as a treatment for disease for millennia. Ayurvedic tradition and treatment has used turmeric as an ingredient in many herbal medicines. Extracts of turmeric contain curcuminoids including curcumin. These compounds have been studied and found to have beneficial effects on cellular health. Curcumin has been shown to posses both anticarcinogenic and antimutagenic effects. Similar to EGCG, curcumin reduces the oxidation of catecholestrogen in vivo, and upregulates both Phase I and Phase II liver enzymes to regulate hormone function in the body. Recent research has show a combination of EGCG and curcumin to have greater effect than the predicted additive effects based on the effects of each alone. Information on turmeric extracts, including curcuminoids and curcumin may be found in the following references, which are incorporated herein in their entirety: Jiang M. C. et al., Curcumin induces apoptosis in immortalized NIH 3T3 and malignant cancer cell lines, Nutr Cancer 1996; (1):111-20; and Subramanian M, et al., Diminution of singlet oxygen-induced DNA damage by curcumin and related antioxidants, Mutation Res 1994; 311:249-55.

Additional elements of the presently disclosed compositions may include fruit flavorings and colorings, such as grape and raspberry in small amounts. Sweeteners, such as momordica fruit may also be included. Components to enhance absorption into the body, such as black or Sichuan pepper extracts may be added. Preservatives, such as sodium benzoate or potassium sorbate may also be included. Substantially pure water, such as deionized water, is also an important ingredient of the liquid mixture.

In one embodiment, the dietary supplement may be provided as a nutritional drink or beverage. The supplement may also be dried into a powder and provided as a freeze dried or spray dried powder, capsule, or tablet. An illustrative beverage supplement is now described in greater detail.

Starting with the fucoidan-containing puree described above, juices or concentrates to provide a high oxygen radical absorbance capacity (ORAC), such as acai fruit, grape, and blueberry are added. Also, fruit flavoring and colorings, such as grape and raspberry; minerals, such as deep sea minerals; sweeteners, such as momordica fruit; pepper for flavor enhancement and to enhance absorption into the body, such as black pepper; preservative, such as sodium benzoate or potassium sorbate; and deionized water are added to the mixture. Next, the mixture is sterilized by pasteurization or other heating techniques. Although pasteurization (at least 87.8° C. or 190° F.) effectively eliminates pathogenic microorganisms, sterilization at higher temperatures maybe needed to eliminate all microorganisms.

In achieving the necessary sterilization, two different sterilization processes are typically used. Using the HTST (high temperature short time) process, the mixture may be raised to about 85° C. (185° F.) for about 20-30 seconds. Alternately, the ultra-high temperature (UHT) process involves raising the temperature of the mixture to about 140.6° C. (285° F.) for about 4-6 seconds. In either process, immediately after the heating step, the temperature is rapidly lowered to at least ambient temperatures of about 21.1-26.7° C. (70-80° F.). Alternately, the mixture may be chilled down to about 4.4° C. (40° F.).

Heating of the mixture may be accomplished by direct or indirect heating. For example, the mixture may be heated by direct contact with steam or indirectly by a selected type of heat exchanger.

The sterilized blend may then be poured into containers, using a hot-fill or cold-fill method. In the hot-fill process, the product is first heated to temperatures for pasteurization, HTST, or UHT. Then it is poured into containers at elevated temperatures to kill any microorganisms inside the container. The use of preservatives, such as sodium benzoate and potassium sorbate are normally used. The pH is usually maintained below 4.4, possibly using acids such as lemon juice or vinegar. After filling, the bottles may be cooled slowly by a water mist. Filling of containers is done by aseptic processing and packaging methods, which are well known in the art.

In the cold-fill process, after pasteurization or sterilization temperatures are reached, the product is immediately cooled to about room temperature prior to bottling, using aseptic processing and packaging techniques. Immediate cooling allows less vitamin degradation and variations in flavor that may be found in the hot-fill process. Thus, in cold-fill processing the flavor may be cleaner and fresher. Preservatives are usually included to control the growth of yeast, molds, and bacteria.

The cold-fill process is compatible with use of high-density polyethylene (HDPE) or polyethylene terephthalate (PET) bottling, so as to not compromise the integrity of the bottle structure. The bottles may be 500 ml bottles, capable of containing about 660 grams per bottle. The size would provide sufficient beverage for 30 days, if a recommended dosage is about 22 grams per day.

Solid dosage forms according to the present invention can be made in the form of powders, tablets, and capsules according to methods well known in the art. For example, powders can be made by drying the fucoidan preparation, and then mixing the dried fucoidan with other dried ingredients. Alternatively, the fucoidan preparation can be mixed with other ingredients, and then the mixture is dried into a powder. Illustrative methods of drying include spray drying and freeze drying. The powder can then be ingested by suspending or dissolving it in a liquid and drinking the resulting suspension or solution. Illustrative liquids that can be used for this purpose include water, juice, and the like. The powder can also be compressed into tablets or loaded into capsules. Tablets or capsules are typically swallowed with water or other liquid. Liquid dietary supplements can also be encapsulated and taken in such a solid dosage form.

### EXAMPLES

The following are examples of the preparation of seaweed to provide a fucoidan puree for use in dietary supplements, and dietary supplement formulations prepared from the fucoidan puree. These examples are merely illustrative and are not meant to be limiting in any way.

The present invention may be embodied in other specific forms without departing from its spirit or essential characteristics. The described embodiments are to be considered in all respects only as illustrative and not restrictive. The scope of the invention is, therefore, indicated by the appended claims, rather than by the description or examples. All changes that come within the meaning and range of equivalency of the claims are to be embraced within their scope.

### Example 1

### Preparation of Fucoidan Puree Composition

Tongan limu moui seaweed was manually harvested, cleaned to remove extraneous material, frozen, and shipped to a processing plant. At the plant, the frozen seaweed was thawed, weighed, and placed in a stainless steel mixer with aqueous buffer and optionally sulfuric acid according to any of the sets of conditions set out in Table 1. The ingredients were then mixed at 50-75 rpm with a medium shear mixer (propeller type). While mixing, the mixture was heated to 37° C. to 95° C. for a selected period of time (usually 5 min to 8 hr). At that point, heating was discontinued, but mixing was continued for 0.5-10 hours to dissipate heat and micronize the seaweed strands. The cooled mixture was then filtered to remove insoluble material, and the filtrate was covered and mixed at room temperature for about 4-72 hours. The pH of the resulting puree was determined to be about pH 2.0 to 4.0, and refractometry typically showed a Brix value of 2-4. The puree comprising partially hydrolyzed fucoidan was then frozen and stored. If sulfuric acid was added during hydrolysis, the partially hydrolyzed fucoidan was sulfonated.

TABLE 1

| | Trials | | | | | | |
|---|---|---|---|---|---|---|---|
| | I | II | III | IV | V | VI | VII |
| pH | 2.0-2.4 | 2.2-2.5 | 2.4-2-7 | 2.6-3.0 | 2.9-3.2 | 3.2-3.6 | 3.6-4.0 |
| sulfuric acid | — | 0.01 N | — | 0.001 N | 0.004 N | — | 0.001 |
| seaweed | 20 wt % | 10 wt % | 25 wt % | 40 wt % | 33 wt % | 15 wt % | 42 wt % |
| temp | 37° C. | 42° C. | 50° C. | 60° C. | 75° C. | 80° C. | 95° C. |
| heating time | 5 hr | 4 hr | 4 hr | 3 hr | 35 min | 20 min | 15 min |
| filtrate | 24 hr, | 16 hr, | 72 hr, | 24 hr, | 48 hr, | 36 hr, | 8 hr, |
| mixing | 37° C. | 37° C. | 22° C. | 22° C. | 22° C. | 22° C. | 22° C. |

### Example 2

### Preparation of Fucoidan Beverage

Fucoidan puree prepared according to the procedure of Example 1 was thawed and then mixed with other ingredients according to the present invention as set out in Tables 2 and 3, where amounts are in parts by weight. These ingredients were blended thoroughly and then sterilized and bottled in by aseptic processing and packaging methods according to any of the conditions set out in Table 4.

TABLE 2

| | Formulation Number | | | | | |
|---|---|---|---|---|---|---|
| | 1 | 2 | 3 | 4 | 5 | 6 |
| fucoidan | 20 | 25 | 30 | 35 | 40 | 45 |
| water | 80 | 75 | 64 | 64.2 | 54.63 | 45.62 |
| grape | | | 6 | | | |
| blueberry | | | | | | 4 |
| acai | | | | 0.5 | | |
| raspberry | | | | | 2.5 | |
| blackberry | | | | | 1.5 | |
| strawberry | | | | | 0.5 | |
| plum | | | | | | |
| orange | | | | | | |
| cherry | | | | | | 4 |
| kiwi | | | | | | |
| currant | | | | | | 1 |
| elderberry | | | | | | |
| black currant | | | | | | |
| cranberry | | | | | | |
| deep sea minerals | | | | | 0.5 | 0.2 |
| momordica | | | | 0.2 | 0.25 | 0.1 |
| sodium benzoate | | | | 0.08 | | 0.05 |
| potassium sorbate | | | | | 0.1 | |
| black pepper | | 0.05 | | 0.01 | 0.02 | |
| Sichuan pepper | | | 0.02 | | | 0.03 |

TABLE 3

| | Formulation Number | | | | | |
|---|---|---|---|---|---|---|
| | 7 | 8 | 9 | 10 | 11 | 12 |
| fucoidan | 0.5 | 8 | 13 | 17 | 19 | 22 |
| water | 86.17 | 85.06 | 83.63 | 76.65 | 72.72 | 69.67 |
| grape | | | 2 | | 5.7 | 5.5 |
| blueberry | | | | 4.2 | | 1.5 |
| acai | | | 0.5 | 0.1 | 0.3 | 0.5 |
| raspberry | | | | | 0.3 | .3 |
| blackberry | | | | | 0.1 | |
| strawberry | | | | 1.0 | 0.8 | |
| plum | | 3.5 | | | | |
| orange | 10 | | | | | |
| cherry | | | | | | |
| kiwi | 3 | | | | 0.4 | |
| currant | | | | | | |
| elderberry | | | 0.3 | | | |
| black | | | | 0.5 | | |
| currant | | | | | | |
| cranberry | | 2.4 | | | | |
| deep sea | | 0.33 | 0.4 | 0.23 | 0.29 | 0.31 |
| minerals | | | | | | |
| momordica | 0.12 | 0.5 | | | 0.17 | 0.16 |
| sodium | 0.12 | | 0.15 | 0.18 | 0.16 | 0.06 |
| benzoate | | | | | | |
| potassium | 0.08 | 0.2 | | | 0.04 | 0.11 |
| sorbate | | | | | | |
| black | 0.005 | 0.01 | | 0.14 | | 0.01 |
| pepper | | | | | | |
| Sichuan | 0.005 | | 0.02 | | 0.015 | |
| pepper | | | | | | |

TABLE 4

| Condition No. | Sterilization | Bottling |
|---|---|---|
| I | 62.8° C., 30 min | hot fill |
| II | 71.7° C., 15 sec | hot fill |
| III | 93.3° C., 10 sec | hot fill |
| IV | 96.0° C., 10 sec | hot fill |
| V | 62.8° C., 30 min | cold fill |
| VI | 140.6° C., 6 sec | cold fill |

### Example 3

About 70 parts by weight of fucoidan puree prepared according to the procedure of Example 1 is mixed with about 99 parts by weight of distilled water, about 20 parts by weight of Concord grape extract, about 2 parts by weight of deep sea minerals, about 1 part by weight of momordica, and about 1 part by weight of black pepper extract. The resulting mixture is spray dried into a powder and packaged for storage and distribution.

### Example 4

The procedure of Example 3 is followed except that the powder is encapsulated in gelatin capsules.

### Example 5

The procedure of Example 3 is followed except that the powder is mixed with selected amounts of diluents, binders, lubricants, disintegrators, colors, flavors, and sweetening agents and then compressed into tablets.

### Skin-Care Products

The present invention advances prior art skin care compositions by providing a skin care composition formulated with fucoidan from seaweed, such as limu moui, kombu, or mozuku. The addition of fucoidan to the skin care composition of the present invention serves to provide significant advantages not found in prior art skin care compositions. The fucoidan-enhanced skin care compositions of the present invention provides many beneficial functions, including providing for anti-aging, and regeneration of cells and tissues; promoting youthfulness; reducing inflammation and the like. In addition, the fucoidan-enhanced skin care compositions of the present invention minimize the visible signs of both biological and environmental aging. That is, the present dietary supplements slow the aging process, assist in regenerating damaged cells and tissues, and promote growth factors in the body. Fucoidan is high in antioxidants that help to fight free radical damage to the body that may lead to cancer. These antioxidants help to fight free radical damage caused by the sun and other changing environmental conditions and elements.

The fucoidan-containing puree described above in connection with dietary supplements may be used in preparing skin care products. Alternately, the mixture may be refrozen in small containers for later processing.

The present invention provides a skin care composition formulated with fucoidan compositions from seaweed, such as the limu moui seaweed plant. The fucoidan compositions are present in selected embodiments from about 0.01 to about 95 percent by weight of the total weight of the composition.

Bases for Skin Care Compositions

Ideally, an ointment base should be nonirritating, nondehydrating, nongreasy, compatible with active ingredients, stable, easily removable with water, absorptive (able to absorb water and/or other liquids), and able to efficiently release the incorporated active ingredients. No ointment base possesses all of these characteristics. Ointments can be classified according to type, based on composition. Such ointment classes include oleaginous bases, absorption bases, emulsion bases, and water-soluble bases.

Oleaginous bases are generally anhydrous, hydrophobic, insoluble in water, and are not water-removable. Oleaginous bases includes the early ointments, which consisted almost entirely of vegetable and animal fats, as well as petroleum hydrocarbons. Fixed oils of vegetable origin include olive, cottonseed, sesame, persic, and other oils. Hydrocarbon bases include ointments prepared from petrolatum or liquid petrolatum with wax or other stiffening agents. Hydrocarbon bases do not become rancid, which is an advantage compared to animal fats and vegetable oils. Another oleaginous base includes silicones, which are synthetic polymers in which the basic structure is an alternating chain of silicon and oxygen atoms (e.g., —O—Si—O—Si—O—Si—). Silicones used in the pharmaceutical and cosmetic industries include dimethylpolysiloxane, methylphenylpolysiloxane, and a stearyl ester of dimethylpolysiloxane, all of which are insoluble in water and are water repellant. Illustrative oleaginous bases are well known in the art, such as Silicone Gibson Base (Example 6) and Vanisil Silicone Ointment (Example 7).

Absorption bases are generally anhydrous, hydrophilic, insoluble in water, and most are not water-removable. These bases have the property of absorbing several times their weight of water and forming emulsions while retaining their ointment-like consistency. Absorption bases vary in their composition, but for the greater part, they are mixtures of animal sterols with petrolatum. Combinations of cholesterol and/or other lanolin fractions with white petrolatum are such absorption bases, and Eucerin and Aquaphor were among the earliest commercial bases of this type. Zopf Emollient Cream (Example 8), Hoch Formula (Example 9), Hydrophilic Petrolatum Base (Example 10), Wool Alcohols Base (Example 11), and Aquabase Ointment (Example 12) are absorption bases described herein. Some commercially available absorption bases include Aquafor (Duke), Polysorb (Fougera), and Nivea Cream (Duke).

Emulsion bases can be either W/O bases, which are hydrous, insoluble in water, and not removable with water and will absorb water, or O/W bases, which are hydrous, insoluble in water, and water-removable and will absorb water. These preparations are solid emulsions, and similar products have long been used as cosmetic creams. The availability of numerous compounds for use as wetting agents, dispersing agents, emulsifiers, penetrants, emollients, detergents, hardeners, preservatives, and the like has given a great deal of flexibility to ointment formulation. Although surface-active agents (i.e., surfactants) may be ionic or nonionic, the nonionic agents are widely used in dermatologic and pharmaceutical preparations. Polysorbate 80 (e.g., Tween 80) and Polyoxyl 40 Stearate represent such surfactants. Nonionic surfactants are generally less toxic and less irritating than ionic surfactants. Other advantages include their virtual neutrality, stability to freezing, stability to electrolytes, and ease of use. In general, the emulsion bases contain an aqueous phase, an emulsifying agent, and an oleaginous phase. The water phase of illustrative emulsion bases typically varies from 10 to 80% by weight of the total base. Glycerin, propylene glycol, or a polyethylene glycol is generally included with the aqueous phase to serve as a humectant, to reduce water loss through evaporation, and to lend a general softness to the creams. The addition of certain alcohols to emulsion base formulas also adds stability to the emulsion and imparts a smooth feel to the skin. Stearyl alcohol, a solid, increases the consistency of the ointment and permits the incorporation of more liquid components. Due to their ability to become hydrated, such alcohols assist in water retention of emulsion bases. The oleaginous phase may contain one or more of the following or similar ingredients: petrolatum, fats, waxes, organic alcohols, polyglycol esters, or other grease-like substances. These substances are emulsified with the aqueous phase through the action of the surfactant. A few such emulsifiers include alkali soaps, alkyl sulfates, amine soaps, polyglycol esters, alkyl aryl sulfates, quaternary ammonium compounds, and the like. These emulsifying compounds aid in the dispersion of the fats and waxes in water and increase the stability of the ointments. Hydrophilic Ointment Base (Example 15), Beeler's Base (Example 16), and U.C.H. Base (Example 17) are illustrative O/W emulsion bases described herein. Commercially available O/W emulsion bases include Cetaphil Cream, Neobase, Unibase, Dermovan, Phorsix Cream, Lubriderm Cream, and Velvachol.

Water-soluble bases are anhydrous, soluble in water, water-removable, and greaseless, and will absorb water. These bases include those bases prepared from polyethylene glycols as well as semisolid preparations containing bentonite, colloidal magnesium aluminum silicate, and sodium alginate. Polyethylene glycol (PEG) compounds 1500, 1540, 4000, and 6000 are of interest in ointment and lotion formulations. PEG 1500 is a soft waxy solid, similar in consistency to petrolatum, with a congealing range of 40° C. to 45° C. PEG 1540 is a solid of consistency of beeswax and is intermediate in physical properties between the 1500 and 4000 PEGs. PEG 4000 has a congealing range of 53° C. to 56° C. and is most useful as a component of being an ointment base for, in addition to the general property of being an emulsifying and dispersing agent, it also adds to the consistency of the base. Both PEG 4000 and PEG 6000 are nonhygroscopic. PEG 6000 is a hard, translucent, waxy solid, and has a congealing range of 58° C. to 62° C.

Glyceryl monostearate is a polyhydric alcohol ester that has been widely used in cosmetic and ointment bases. It has a high melting point (56° C. to 58° C.) and is a good emulsifying agent. Glyceryl monostearate emulsions generally contain high water phases, usually above 60% by weight. It has the disadvantage of being incompatible with acids. Glyceryl Monostearate Base (Example 27) is described herein.

Cellulose derivatives, such as methylcellulose and hydroxyethyl cellulose, form colloidal solutions that resemble gums and mucilages, but are not as vulnerable to fungal or bacterial attack. Methylcellulose is dispersible in cold water, but in concentrated solutions will coagulate upon heating. Hydroxyethyl cellulose is more soluble at elevated temperatures so that viscosity of aqueous solutions decreases slightly on warming. It is a good protective colloid for aqueous dispersions of oils, waxes, and pigments. Sodium carboxymethylcellulose is another cellulose derivative frequently referred to as carboxymethyl cellulose or CMC. It is an anionic compound and thereby can be used as a thickening or stabilizing agent for suspensions and for ointments of the emulsion type where the emulsifying agent is anionic or nonionic. Any of these cellulose derivatives can be used to stabilize ointment formulas, and they are commercially available in various viscosity types and with various degrees of substitution.

Sodium alginate is a hydrophilic colloid that is compatible with small amounts of alcohol, glycerin, polyglycols, wetting agents, and solutions of alkali carbonates. It functions satisfactorily under acid or alkaline conditions within the pH range of 4.5-10. It is possible to make sodium alginate solutions into semi-firm or firm gels by the addition of small amounts of soluble calcium salts, i.e., calcium gluconate, calcium tartrate, and calcium citrate. Ions of the alkaline earth metals will thicken or gelatinize sodium alginate solutions when present in low concentrations, while at high concentrations they will precipitate them. A 2.5% solution of sodium alginate is a satisfactory inert diluent for greaseless and other types of ointments.

Bentonite, a colloidal hydrated aluminum silicate, is insoluble in water, but when mixed with 8 to 10 parts of water it swells to produce a slightly alkaline gel resembling petrolatum. The consistency of the product may be regulated by varying the amounts of water added. Ointments prepared from bentonite and water alone are found to be slightly drying and unstable upon standing, but addition of a humectant, such as glycerin or sorbitol, in amounts up to about 10% by weight will retard this action. Ointments prepared from bentonite do not encourage mold growth, and they have the advantage of not spreading to the hair when applied to the scalp.

Colloidal magnesium aluminum silicate (e.g., Veegum®, R.T. Vanderbilt Company, Inc.) is an inorganic emulsifier, suspending agent, and thickener. Dispersions are slightly alkaline and are compatible with about 20 to 30% ethyl alcohol, isopropyl alcohol, acetone, and similar solvents. Glycols, such as glycerin and propylene glycol, are compatible at 40 to 50% concentrations.

Carbopol 934 (carboxypolymethylene) is an acid polymer that disperses readily in water to yield an acid solution of low viscosity. When the acid solution is neutralized with a suitable base, such as sodium bicarbonate, sodium hydroxide, or the like, a clear, stable gel results. Carbopol 934 is inert physiologically and is neither a primary irritant nor a sensitizer. The thickening efficiency of Carbopol 934 can be used in the preparation of such pharmaceuticals as creams, ointments, lotions, suspensions, and emulsions.

The skin care compositions of the present invention can also contain fragrances, proteins, colorants or coloring agents, lipids, vitamins, botanical extracts, lipids, glycolipids, polymers, and copolymers, and the like, as are generally known in the art of making skin care products. The Cosmetic, Toiletry, and Fragrance Association's International Cosmetic Ingredient Dictionary and Handbook is an excellent source of information concerning such ingredients.

As used herein, “colorants” or “coloring agents” are agents that give skin care compositions a more pleasing appearance, and in addition help the manufacturer to control the product during its preparation and help the user to identify the product. Any of the approved certified water-soluble FD&C dyes, mixtures thereof, or their corresponding lakes may be used to color skin care compositions. A color lake is the combination by adsorption of a water-soluble dye to a hydrous oxide of a heavy metal, resulting in an insoluble form of the dye.

The skin care compositions of the present invention are applied to the skin in amounts selected by the user. The compositions are dispensed from appropriate containers and are generally manually applied to the skin, as is well known in the art.

### Example 6

### Silicone Gibson Base

The following formula illustrates a silicone base that can be used in a cream or lotion according to the present invention. Silicone Gibson base comprises 15 parts by weight of cetyl alcohol, 1 parts by weight of sodium lauryl sulfate, 40 parts by weight of dimethylpolysiloxane polymer (1000 cps), 43 parts by weight purified water, 0.25 parts by weight methylparaben, and 0.15 parts by weight propylparaben. The aqueous mixture of the sodium lauryl sulfate and the parabens is warmed to 75° C., and then it is slowly added to warmed (25° C.) cetyl alcohol-silicone mixture. The resulting mixture is stirred until it congeals.

### Example 7

### Vanisil Silicone Ointment Base

The following formula illustrates a silicone base that can be used in a cream or lotion according to the present invention. Vanisil silicone ointment base comprises 10 parts by weight stearic acid, 2 parts by weight synthetic Japan wax, 20 parts by weight dimethylpolysiloxane polymer (1000 cps), 0.5 parts by weight potassium hydroxide, 0.025 parts by weight methylparaben, 0.015 parts by weight propylparaben, and 67.5 parts by weight distilled water.

### Example 8

### Zopf Emollient Cream

The following formula illustrates a W/O emulsion absorption base that can be used according to the present invention. Zopf emollient cream comprises 41 parts by weight of white petrolatum, 3 parts by weight of microcrystalline wax, 10 parts by weight of fluid lanolin, 4.75 parts by weight sorbitan monooleate, 0.25 parts by weight of polysorbate 80, and 41 parts by weight purified water. The aqueous dispersion of sorbitan monooleate and polysorbate 80 is warmed to 75° C. and then slowly added to the melted wax, white petrolatum, and fluid lanolin. The resulting mixture is stirred until it congeals.

### Example 9

### Hoch Formula

The following formula illustrates an O/W emulsion absorption base that can be used according to the present invention. Hoch formula comprises phase A comprising 5 parts by weight of fluid lanolin, 35 parts by weight of castor oil, 2 parts by weight of sorbitan monostearate, 36.7 parts by weight of mineral oil, 4 parts by weight of stearic acid, and 0.2 parts by weight of propylparaben; and phase B comprising 1 parts by weight of polyethylene 20 sorbitan monostearate, 0.9 parts by weight of triethanolamine, 0.2 parts by weight of methylparaben, and 15 parts by weight of purified water. Phase A is heated to 78° C., and phase B is heated to 70° C. Then, phase B is added to phase A and the resulting mixture is stirred until it cools to 25° C.

### Example 10

### Hydrophilic Petrolatum Base

The following formula illustrates an absorption base that can be used according to the present invention. Hydrophilic petrolatum base comprises 30 parts by weight of cholesterol, 30 parts by weight of stearyl alcohol, 80 parts by weight of white wax, and 860 parts by weight of white petrolatum. The stearyl alcohol, white wax, and white petrolatum are melted together on a steam bath, and then the cholesterol is added and stirred into the mixture until the cholesterol completely dissolves. The mixture is then removed from the bath and stirred until it congeals.

### Example 11

### Wool Alcohols Base

The following formula illustrates an absorption base that can be used according to the present invention. Wool alcohols ointment base comprises 60 parts by weight wool alcohols, 240 parts by weight hard paraffin, 100 parts by weight white or yellow soft paraffin, and 600 parts by weight liquid paraffin. The ingredients are mixed together and stirred until cold.

### Example 12

### Aquabase Ointment

The following formula illustrates an absorption base that can be used according to the present invention. Aquabase ointment comprises 30 parts by weight of cholesterol, 30 parts by weight of cottonseed oil, and 940 parts by weight of white petrolatum. The white petrolatum and cottonseed oil are heated to 145° C. and then removed from the heat. The cholesterol is then added and stirred until it is almost congealed. Then the ointment is placed in suitable containers.

### Example 13

### Emulsion Base

The following formula illustrates an emulsion base that can be used according to the present invention. Many dermatologic and cosmetic preparations contain amine soaps as emulsifying agents. These anionic emulsifiers are advantageous as compared to sodium and potassium soaps because they yield emulsions having a relatively low pH of about 8.0. Triethanolamine is generally used, along with a fatty acid, to produce the fatty acid amine soap. Triethanolamine usually contains small amounts of ethanolamine and diethanolamine. It combines stoichiometrically with fatty acids. Semisolid O/W bases containing triethanolamine soaps are generally prepared by dissolving the triethanolamine in water and then adding this solution to the oil phase with stirring. A typical formula for such a base comprises 18 parts by weight stearic acid, 4 parts by weight of cetyl alcohol, 2 parts by weight of triethanolamine, 5 parts by weight of glycerin, and 71 parts by weight of distilled water.

### Example 14

### Coal Tar Ointment Base

The following formula illustrates an emulsion base that can be used according to the present invention. Coal tar ointment base contains a surfactant, i.e., polysorbate 80, which serves the dual purpose of a dispersing agent and aiding in removal of the ointment from the skin. Coal tar ointment comprises 10 parts by weight coal tar, 5 parts by weight polysorbate 80, and 985 parts by weight zinc oxide paste. The coal tar is blended with the polysorbate 80, and this blend is then mixed with the zinc oxide paste.

### Example 15

### Hydrophilic Ointment Base

The following formula illustrates an emulsion base that can be used according to the present invention. Hydrophilic ointment base comprises 0.25 parts by weight methylparaben, 0.15 parts by weight propylparaben, 10 parts by weight sodium lauryl sulfate, 120 parts by weight propylene glycol, 250 parts by weight stearyl alcohol, 250 parts by weight white petrolatum, and 370 parts by weight water. The stearyl alcohol and white petrolatum are melted on a steam bath and warmed to about 75° C. The other ingredients, previously dissolved in the water, are warmed to 75° C. and then added with stirring until the mixture congeals.

### Example 16

### Beeler's Base

The following formula illustrates an O/W emulsion base that can be used according to the present invention. Beeler's base comprises 15 parts by weight cetyl alcohol, 1 parts by weight white wax, 10 parts by weight propylene glycol, 2 parts by weight sodium lauryl sulfate, and 72 parts by weight water. The cetyl alcohol and white wax are melted in the propylene glycol on a water bath, and the resulting mixture is heated to about 65° C. The sodium lauryl sulfate is dissolved in the water and also heated on water bath to about 65° C. The oil phase is slowly added to the well-stirred water phase, and stirring is continued on the water bath for about 10 min. The emulsion is then removed from the water bath and stirring is continued to the point of congealing.

### Example 17

### U.C.H. Base

The following formula illustrates an emulsion base that can be used according to the present invention. U.C.H. base comprises 6.4 parts by weight cetyl alcohol, 5.4 parts by weight stearyl alcohol, 1.5 parts by weight sodium lauryl sulfate, 14.3 parts by weight white petrolatum, 21.4 parts by weight mineral oil, and 50 parts by weight water. The alcohols are melted together over a water bath at 65° C., then the sodium lauryl sulfate is add with stirring. Next the white petrolatum and the mineral oil are added with continued heating of the mixture until it is completely melted. This mixture is then cooled to room temperature and the water is added with constant mixing to result in the emulsion.

### Example 18

### Base A

The following formula illustrates an anhydrous emulsifiable solid mixture. Anhydrous solid mixture A is made by melting together 53 parts by weight of stearyl alcohol, 7 parts by weight of cetyl alcohol, 38.6 parts by weight of PEG 400, and 1.4 parts by weight of sodium lauryl sulfate. These ingredients are melted and stirred vigorously until completely solidified. Stirring is continued to insure complete mixing of the ingredients and for the production of a granular product. Base A is made by melting 50 parts by weight of the granular solid mixture A, heating it to 70-75° C., and then adding it to 50 parts by weight of an aqueous mixture at the same temperature. The mixture is stirred until the emulsion begins to solidify and cools to 40° C. The resulting base is a white, semisolid O/W emulsion of ointment-like consistency. It is non-greasy and washable with water. The emulsion is stable up to 55-60° C., exhibits a good sheen, and exhibits good lubricity when applied to skin.

### Example 19

### Base B

The following formula illustrates an anhydrous emulsifiable solid mixture. Anhydrous solid mixture B is made by melting together 64.7 parts by weight of stearyl alcohol, 8.6 parts by weight of cetyl alcohol, 13 parts by weight of PEG 1000 monostearate, 8.7 parts by weight of PEG 1540, and 5 parts by weight of anhydrous lanolin. These ingredients are melted and stirred vigorously until completely solidified. Stirring is continued to insure complete mixing of the ingredients and for the production of a granular product. Base B is made by melting 40 parts by weight of the granular solid mixture B, heating it to 70-75° C., and then adding it to 60 parts by weight of an aqueous mixture at the same temperature. The mixture is stirred until the emulsion begins to solidify and cools to 40° C. The resulting base is a white, semisolid O/W emulsion of ointment-like consistency. It is non-greasy and washable with water. The emulsion is stable up to 55-60° C. and exhibits good lubricity when applied to skin.

### Example 20

### Aqueous Cream Base

The following formula illustrates an emulsion base that can be used according to the present invention. Aqueous cream base is an emulsion base prepared from 30% by weight of emulsifying ointment and 70% by weight of water. Emulsifying ointment comprises 30 parts by weight emulsifying wax, 20 parts by weight liquid paraffin, and 50 parts by weight white soft paraffin. Emulsifying wax comprises 90 parts by weight cetostearyl alcohol, 10 parts by weight sodium lauryl sulfate, and 4 parts by weight purified water.

### Example 21

### Polyethylene Glycol Ointment Base

The following formula illustrates a water-soluble base that can be used according to the present invention. Polyethylene glycol ointment base comprises 400 parts by weight of PEG 4000 and 600 parts by weight of PEG 400. The two ingredients are heated on a water bath to 65° C., and then the mixture is allowed to cool with stirring until it congeals. If a firmer preparation is desired, up to 100 parts by weight of the PEG 400 can be replaced with an equal amount of PEG 4000. If 6-25% by weight of an aqueous solution is to incorporated in this polyethylene ointment, 50 parts by weight of the PEG 4000 is replaced with an equal amount of stearyl alcohol.

### Example 22

### Base G

The following formula illustrates a water-soluble base that can be used according to the present invention. The addition of an ester of polyethylene glycol to a polyethylene glycol ointment yields a water-removable, emulsifiable ointment base. An illustrative emulsifiable glycol ointment base (Base G) of this type comprises 26 parts by weight polyethylene glycol 400 monostearate, 37 parts by weight PEG 400, and 37 parts by weight PEG 4000. The glycols are mixed and melted at about 65° C. This mixture is then stirred while cooling to about 40° C. The polyethylene glycol 400 monostearate is melted at about 40° C. and then added to the liquid glycol mixture with stirring until a uniform ointment is obtained. Water (10-15% by weight) can be incorporated into Base G.

### Example 23

### Base III

The following formula illustrates a water-soluble base that can be used according to the present invention. Surfactants and water can be added to a polyethylene glycol ointment without impairing the water removability of the base. Base III represents a typical formula of this type: 50 parts by weight PEG 4000, 40 parts by weight PEG 400, 1 parts by weight sorbitan monopalmitate, and 9 parts by weight water. The sorbitan monopalmitate and the polyethylene glycols are warmed together on a water bath to 70° C. and the water heated to the same temperature is then added. The emulsion is stirred until it congeals.

### Example 24

### Modified Landon-Zopf Base

The following formula illustrates a water-soluble base that can be used according to the present invention. Modified Landon-Zopf base comprises 20 parts by weight PEG 4000, 34 parts by weight stearyl alcohol, 30 parts by weight glycerin, 15 parts by weight water, and 1 parts by weight sodium lauryl sulfate. The PEG 4000, stearyl alcohol, and glycerin are heated on a water bath to 75° C. This mixture is then added in small quantities with stirring to the water, which contains the sodium lauryl sulfate and has also been heated to 75° C. Moderate stirring is continued until the base has congealed.

### Example 25

### Canadian Base

The following formula illustrates a water-soluble base that can be used according to the present invention. Canadian base comprises 11.2 parts by weight PEG 4000, 20.8 parts by weight stearyl alcohol, 17 parts by weight glycerin, 0.6 parts by weight sodium lauryl sulfate, and 50.4 parts by weight water. The PEG 4000, stearyl alcohol, and glycerin are heated on a water bath to 70° C. The water, which contains the sodium lauryl sulfate and has been previously heated to 70° C., is added and the mixture is stirred until the base congeals.

### Example 26

### Base IV

The following formula illustrates a water-soluble base that can be used according to the present invention. Base IV comprises 42.5 parts by weight PEG 4000, 37.5 parts by weight PEG 400, and 20 parts by weight 1,2,6-hexanetriol. The PEG 4000 is heated with the 1,2,6-hexanetriol is heated on a water bath to 60-70° C. This mixture is added to the PEG 400 at room temperature with vigorous stirring. The, occasional stirring is continued until solidification takes place.

### Example 27

### Glyceryl Monostearate Base

The following formula illustrates a water-soluble base that can be used according to the present invention. Glyceryl monostearate base comprises 10 parts by weight mineral oil, 30 parts by weight white petrolatum, 10 parts by weight glyceryl monostearate S. E., 5 parts by weight cetyl alcohol, 5 parts by weight glycerin, and 40 parts by weight water.

### Example 28

### Lubricating Jelly Base

The following formula illustrates a water-soluble base that can be used according to the present invention. Lubricating jelly base comprises 1 g methocel 90 HC 4000, 0.3 g carbopol 934, sodium hydroxide qs pH 7.0, 20 ml propylene glycol, 0.15 g methylparaben, and purified water qs 100 parts by weight. The methocel is added slowly to 40 ml of hot water (80-90° C.) and agitated for 5 min. After cooling, the solution is refrigerated overnight. The carbopol 934 is dissolved in 20 ml of water, and 1% sodium hydroxide is added slowly with cautious stirring to avoid incorporation of air, until a pH of 7.0 is obtained, and then water is added to a total volume of 40 ml. The methylparaben is dissolved in the propylene glycol. Finally the methocel, carbopol, and methylparaben solutions are mixed cautiously to avoid incorporation of air.

### Example 29

### Universal O/W Ointment Base

The following formula illustrates a water-soluble base that can be used according to the present invention. Universal O/W ointment base comprises 0.05 parts by weight calcium citrate, 3 parts by weight sodium alginate, 0.20 parts by weight methylparaben, 45 parts by weight glycerin, and sufficient distilled water to make a total of 100 parts by weight. The calcium citrate and the methylparaben are dissolved in the water. The glycerin is mixed with the sodium alginate to form a smooth paste. The aqueous mixture is added to the paste and is stirred until a smooth, stiff preparation is obtained. The base is then set aside for several hours until thickening is complete.

### Example 30

### Hollander and McClanahan Base

The following formula illustrates a water-soluble base that can be used according to the present invention. Hollander and McClanahan base comprises 32 parts by weight petrolatum, 13 parts by weight bentonite, 0.5 parts by weight sodium lauryl sulfate, 54 parts by weight water, and 0.1 parts by weight methylparaben.

### Example 31

### MGH Ointment Base

The following formula illustrates a water-soluble base that can be used according to the present invention. MGH ointment base comprises 15 parts by weight polyethylene glycol 200 monostearate, 2.5 parts by weight colloidal magnesium stearate silicate (Veegum), 1 parts by weight polysorbate 80, 0.1 parts by weight methylparaben, and 81.4 parts by weight purified water.

### Example 32

### Lotion Base

The following formula illustrates a water-soluble base that can be used according to the present invention. Lotion base comprises 1 parts by weight Veegum, 0.85 parts by weight sodium carboxymethylcellulose, 90.15 parts by weight water, 3 parts by weight glycerin, and 5 parts by weight dioctyl sodium sulfosuccinate (1% solution). All the dry ingredients are mixed with water and glycerin in a blender for 1 min. The mixture is then removed from the blender and the dioctyl sodium sulfosuccinate is added.

### Example 33

### Cold Cream Base

The following formula illustrates a cold cream according to the present invention. A cold cream base comprises 6 parts by weight spermaceti, 6 parts by weight beeswax, 10 parts by weight Carbopol 934, 4.75 parts by weight sodium carbonate, 5 parts by weight rose water, 0.02 parts by weight rose oil, 56 parts by weight expressed almond oil, and 20 parts by weight distilled water.

### Example 34

### Hand Lotion Base

The following formula illustrates a hand lotion according to the present invention. A hand lotion base comprises 24.75 ml propylene glycol, 1 ml triethanolamine, 12 ml water, 1.5 g oleic acid, 10.5 g polyethylene glycol 400 monostearate, 10 ml silicone fluid D.C. 200, and 50 g carbopol 934 2% mucilage.

### Example 35

### White Lotion Base

White lotion base comprises 40 parts by weight zinc sulfate, 40 parts by weight sulfurated potash, and sufficient purified water to make 1000 parts by weight. The zinc sulfate and the sulfurated potash are dissolved separately, each in 450 parts by weight of purified water, and then each solution is filtered. The sulfurated potash solution is then added slowly to the zinc sulfate solution with constant stirring. Then the remainder of the water is added, and the lotion is mixed.

The subject matter claimed is:

1. A composition of matter for delivery of partially hydrolyzed and/or sulfonated fucoidan comprising said fucoidan formulated as nanoparticles.

2. The composition of claim 1, further comprising clustered water.

3. The composition of claim 1, further comprising an antioxidant selected from the group consisting of superoxide dismutase, astaxanthin, curcumin, curcuminoids, vitamin E, raspberry, blueberry, pomegranate, tocopherols, green tea, white tea, dark chocolate, chocolate, cocoa, spirulina, bromelain, vitamin C, rutin, grape seed extract, pycnogenols, oligomeric proanthocyanidins, anthocyanidins, procyanidins, selenium, beta-carotene, zinc, bilberry, cranberry, polyphenols, flavones, strawberry, ellagic acid, coumarin, ferulic acid, resveratrol, alpha-lipoic acid, tomatoes, avocados, broccoli, lycopene, lutein, vitamin A, folic acid, folates, carotenoids, olive leaf extract, ground cloves, ground cinnamon, oregano, blackberry, black currant, polyphenolics, bioflavonoids, flavonoids, flavanols, catechols, goji, tamarind, mangosteen, xanthones, tart cherries, cherries, asparagus, glutathione, catechins, epicatechins, plums, ruby queen plum, kiwi fruit, Ganoderma lucidum, thiols, onions, apples, red cabbage, star fruit, carambola, white pine bark extract, N-acetyl cysteine, citrus, beta-cryptoxanthin, and mixtures thereof.

4. The composition of claim 1, further comprising a member selected from the group consisting of glucosamine sulfate, glucosamine HCl, glucosamine phosphate, acetyl glucosamine, shark cartilage, chondroitin sulfate, galactolipids, wool keratin protein extract, keratin extract, hyaluronic acid, stinging nettle, glucomannan, type 11 collagen, collagen hydrolysate, and mixtures thereof.

5. The composition of claim 1, further comprising a member selected from the group consisting of noni, mangosteen, and mixtures thereof.

6. The composition of claim 1, further comprising a saccharide selected from the group consisting of glucose, sucrose, fructose, and mixtures thereof.

7. The composition of claim 1, further comprising a member selected from the group consisting of α-amino acids, salts of α-amino acids, and esters of α-amino acids.

8. The composition of claim 1, further comprising a member selected from the group consisting of capsican, lycopene, lutein, perillyl oil, cranberry, curcumin, turmeric, and mixtures thereof.

9. A composition of matter for delivery of partially hydrolyzed and/or sulfonated fucoidan wherein said composition comprises a mixture of said fucoidan and clustered water.

10. The composition of claim 9, further comprising an antioxidant selected from the group consisting of superoxide dismutase, astaxanthin, curcumin, curcuminoids, vitamin E, raspberry, blueberry, pomegranate, tocopherols, green tea, white tea, dark chocolate, chocolate, cocoa, spirulina, bromelain, vitamin C, rutin, grape seed extract, pycnogenols, oligomeric proanthocyanidins, anthocyanidins, procyanidins, selenium, beta-carotene, zinc, bilberry, cranberry, polyphenols, flavones, strawberry, ellagic acid, coumarin, ferulic acid, resveratrol, alpha-lipoic acid, tomatoes, avocados, broccoli, lycopene, lutein, vitamin A, folic acid, folates, carotenoids, olive leaf extract, ground cloves, ground cinnamon, oregano, blackberry, black currant, polyphenolics, bioflavonoids, flavonoids, flavanols, catechols, goji, tamarind, mangosteen, xanthones, tart cherries, cherries, asparagus, glutathione, catechins, epicatechins, plums, ruby queen plum, kiwi fruit, Ganoderma lucidum, thiols, onions, apples, red cabbage, star fruit, carambola, white pine bark extract, N-acetyl cysteine, citrus, beta-cryptoxanthin, and mixtures thereof.

11. The composition of claim 9, further comprising a member selected from the group consisting of glucosamine sulfate, glucosamine HCl, glucosamine phosphate, acetyl glucosamine, shark cartilage, chondroitin sulfate, galactolipids, wool keratin protein extract, keratin extract, hyaluronic acid, stinging nettle, glucomannan, type 11 collagen, collagen hydrolysate, and mixtures thereof.

12. The composition of claim 9, further comprising a member selected from the group consisting of noni, mangosteen, and mixtures thereof.

13. The composition of claim 9, further comprising a saccharide selected from the group consisting of glucose, sucrose, fructose, and mixtures thereof.

14. The composition of claim 9, further comprising a member selected from the group consisting of α-amino acids, salts of α-amino acids, and esters of α-amino acids.

15. The composition of claim 9, further comprising a member selected from the group consisting of capsican, lycopene, lutein, perillyl oil, cranberry, curcumin, turmeric, and mixtures thereof.

16. A composition of matter comprising mixtures or compounds of partially hydrolyzed and/or sulfonated fucoidan and a member selected from the group consisting of (a) a cell signaling agent comprising bitter orange, caffeine, taurine, green coffee bean, or mixtures thereof; (b) a heart strengthening agent comprising wolfberry; and (c) equol.

17. The composition of claim 16, further comprising clustered water.

18. The composition of claim 16, wherein said composition is formulated as nanoparticles.

19. The composition of claim 16, further comprising an antioxidant selected from the group consisting of superoxide dismutase, astaxanthin, curcumin, curcuminoids, vitamin E, raspberry, blueberry, pomegranate, tocopherols, green tea, white tea, dark chocolate, chocolate, cocoa, spirulina, bromelain, vitamin C, rutin, grape seed extract, pycnogenols, oligomeric proanthocyanidins, anthocyanidins, procyanidins, selenium, beta-carotene, zinc, bilberry, cranberry, polyphenols, flavones, strawberry, ellagic acid, coumarin, ferulic acid, resveratrol, alpha-lipoic acid, tomatoes, avocados, broccoli, lycopene, lutein, vitamin A, folic acid, folates, carotenoids, olive leaf extract, ground cloves, ground cinnamon, oregano, blackberry, black currant, polyphenolics, bioflavonoids, flavonoids, flavanols, catechols, goji, tamarind, mangosteen, xanthones, tart cherries, cherries, asparagus, glutathione, catechins, epicatechins, plums, ruby queen plum, kiwi fruit, Ganoderma lucidum, thiols, onions, apples, red cabbage, star fruit, carambola, white pine bark extract, N-acetyl cysteine, citrus, beta-cryptoxanthin, and mixtures thereof.

20. The composition of claim 16, further comprising a member selected from the group consisting of capsican, lycopene, lutein, perillyl oil, cranberry, curcumin, turmeric, and mixtures thereof.
